(19) United States
(12) Patent Application Publication
(10) Pub. No.: US 2026/0112496 A1
(43) Pub. Date: Apr. 23, 2026

(54) ANATOMICAL FEATURE-BASED MEDICAL PREDICTION

(71) Applicant: Soliish Inc., Fairfax, VA (US)

(72) Inventors: Jatin Maniar, Fairfax, VA (US); Narayanan Ramanathan, Aldie, VA (US); Ikshvanku Barot, Suffolk, VA (US); Hitesh Kalra, Cupertino, CA (US)

(21) Appl. No.: 19/331,908

(22) Filed: Sep. 17, 2025

Related U.S. Application Data
(60) Provisional application No. 63/695,533, filed on Sep. 17, 2024.

(51) Int. Cl. G16H 50/30; G06T 7/00; G06T 7/11; G06T 7/33; G16H 10/20; G16H 10/60; G16H 30/20

(52) U.S. Cl. CPC G16H 50/30; G06T 7/0012; G06T 7/11; G06T 7/33; G16H 10/20; G16H 10/60; G16H 30/20; G06T 2207/20081; G06T 2207/30036; G06T 2207/30201

(57) ABSTRACT

Methods, systems, and apparatus, including computer programs encoded on computer storage media, for anatomical feature-based medical predictions. One of the methods includes capturing a set of images depicting at least a portion of a subject body. The set of images are processed to extract a subset of images that satisfy one or more feature thresholds, such as thresholds related to anatomical pose or image quality. One or more target anatomical structures, such as craniofacial or oral cavity structures, are detected within the subset of images. One or more anatomical features are generated for the target anatomical structures, for instance, by fitting a three-dimensional model to the structures and computing geometric metrics. Using the generated anatomical features and stored medical data, a prediction is generated indicating a likelihood of a medical condition, such as obstructive sleep apnea or a comorbid condition, which can be transmitted to a device.

### CROSS-REFERENCE TO RELATED APPLICATIONS

This application claims the benefit of U.S. Provisional Application No. 63/695,533, filed Sep. 17, 2024, the contents of which are incorporated in its entirety by reference herein.

### TECHNICAL FIELD

This specification relates to detection of medical conditions using one or more trained machine learning models.

### BACKGROUND

Various computer-based systems exist for processing visual data, such as images or video streams. These systems can leverage techniques from fields like computer vision and machine learning to identify features or patterns within the data. For instance, some systems are configured to analyze images to detect specific objects or to characterize attributes of a depicted scene. In some applications, machine learning models are trained on datasets of images to perform classification or regression tasks based on the visual information contained within those images. The processing of such data can occur on various computing devices, including servers or personal electronic devices.

### SUMMARY

This specification describes technologies for predicting medical conditions using biometric data. These technologies generally involve capturing one or more images of a subject (e.g., a human or a non-human animal), such as images depicting the subject's craniofacial region or oral cavity, and extracting a subset of those images for analysis based on one or more quality or content criteria. Anatomical features are then generated from the extracted images, for instance, by fitting a two-dimensional or three-dimensional model to the depicted anatomical structures and calculating metrics such as distances, angles, or volumes. These generated anatomical features are compared with stored medical data indicative of one or more targeted conditions, such as Obstructive Sleep Apnea (OSA) or comorbid neurodegenerative or cardiovascular diseases, to generate a prediction regarding the subject's health. The process may be performed using a personal computing device, such as a smartphone, with or without server-based processing aid.

In general, one innovative aspect of the subject matter described in this specification can be embodied in methods that include the actions of capturing a first set of images depicting at least a portion of an oral cavity region of a subject body; detecting a set of image features associated with the oral cavity region within the first set of images; accessing one or more stored feature thresholds; extracting, from the first set of images and using the stored feature thresholds, a second set of images that satisfy one or more of the stored feature thresholds; upon extracting the second set of images, detecting a set of regions within the second set of images that represent one or more target anatomical structures within the oral cavity region of the subject body; generating one or more anatomical features for at least one region of the set of regions that represent the target anatomical structures of the subject body; accessing one or more stored obstructive sleep apnea features that indicate obstructive sleep apnea; generating a prediction indicating a likelihood that the subject body suffers from obstructive sleep apnea using (i) the anatomical features for the at least one region of the oral cavity of the subject body and (ii) the stored obstructive sleep apnea features; and transmitting a signal indicating the generated prediction of obstructive sleep apnea to a connected device. Other embodiments of this aspect include corresponding computer systems, apparatus, and computer programs recorded on one or more computer storage devices, each configured to perform the actions of the methods.

The foregoing and other embodiments can each optionally include one or more of the following features, alone or in combination. In particular, one embodiment includes all the following features in combination. Feature 1: Capturing the first set of images depicting at least the portion of the oral cavity region of the subject body includes capturing the first set of images using a handheld image capture device. Feature 2: Capturing the first set of images using the handheld image capture device includes capturing the first set of images using a smartphone. Feature 3: Capturing an image of a surface region of a head of the subject body, where the method includes detecting a second set of image features associated with the head of the subject body, where accessing the one or more stored feature thresholds includes accessing a stored feature threshold corresponding to a head feature, where extracting the second set of images that satisfy one or more of the stored feature thresholds includes extracting (i) a first image that indicates a first feature that satisfies a first stored feature threshold associated with the oral cavity region of the subject body and (ii) a second image that indicates a second feature that satisfies a second stored feature threshold associated with the head of the subject body. Feature 4: Actions include generating a three-dimensional model of at least the surface region of the head of the subject body, where detecting the set of regions within the second set of images includes detecting the set of regions using the generated three-dimensional model. Feature 5: Detecting a lower jaw region and an upper jaw region using the generated three-dimensional model. Feature 6: Detecting a subregion of the oral cavity region of the subject body, where the subregion includes at least one of an upper teeth region, a lower teeth region, a back of the mouth region, or a tongue region. Feature 7: Accessing an axis of symmetry feature threshold indicating a degree of symmetry within one or more subregions of the oral cavity region of the subject body, where extracting the second set of images that satisfy one or more of the stored feature thresholds includes extracting an image that includes a subregion within a threshold percentage of vertical alignment. Feature 8: Using an image segmentation library to identify a plurality of segmented regions within the oral cavity region, and where extracting the second set of images that satisfy one or more of the stored feature thresholds includes extracting an image in which the image segmentation library identifies at least three well-defined regions selected from a group consisting of an upper teeth region, a lower teeth region, a back of the mouth region, and a tongue region, where a vertical axis of symmetry for each of the at least three well-defined regions is substantially in alignment. Feature 9: Further including screening for one or more medical conditions comorbid with obstructive sleep apnea, where screening for the one or more medical conditions includes capturing a second set of images depicting a motor task performed by at least one hand of the subject body; detecting, from the second set of images, a plurality of hand features corresponding to movements of the at least one hand; generating one or more movement metrics based on the plurality of hand features; and generating a prediction indicating a likelihood of a neurodegenerative condition based on the one or more movement metrics. Feature 10: The motor task includes repeatedly touching a tip of an index finger to a tip of a thumb, and where generating the one or more movement metrics includes calculating a frequency of contact between the tip of the index finger and the tip of the thumb. Feature 11: Further including screening for a cardiovascular condition comorbid with obstructive sleep apnea, where screening for the cardiovascular condition includes detecting, from the first set of images, a region corresponding to an earlobe of the subject body; analyzing the region corresponding to the earlobe to detect a presence of a diagonal crease; and generating a prediction indicating an increased risk of coronary artery disease in response to detecting the presence of the diagonal crease. Feature 12: Further including screening for a potential past stroke comorbid with obstructive sleep apnea, where screening for the potential past stroke includes identifying a plurality of fiducial landmarks on a face of the subject body within the first set of images; calculating a degree of facial symmetry using the plurality of fiducial landmarks; and generating a prediction indicating a likelihood of a past stroke in response to determining that the degree of facial symmetry is below a symmetry threshold.

Another innovative aspect of the subject matter described in this specification can be embodied in methods that include the actions of capturing a first set of images depicting at least a portion of a subject body; detecting a set of image features within the first set of images; accessing one or more stored feature thresholds; extracting, from the first set of images and using the stored feature thresholds, a second set of images that satisfy one or more criteria corresponding to the stored feature thresholds; upon extracting the second set of images, detecting a set of regions within the second set of images that represent one or more target anatomical structures of the subject body; generating one or more anatomical features for at least one region of the set of regions that represent the target anatomical structures of the subject body; accessing one or more stored medical condition features associated with one or more medical conditions; generating a predicted medical condition of the subject body, from among the one or more medical conditions, using the anatomical features and the stored medical condition features; and transmitting a signal indicating the generated predicted medical condition to a connected device. Other embodiments of this aspect include corresponding computer systems, apparatus, and computer programs recorded on one or more computer storage devices, each configured to perform the actions of the methods.

The foregoing and other embodiments can each optionally include one or more of the following features, alone or in combination. In particular, one embodiment includes all the following features in combination. Feature 1: Actions include generating a three-dimensional model of at least the portion of the subject body, where detecting the set of regions within the second set of images includes detecting the set of regions using the generated three-dimensional model. Feature 2: Detecting the set of regions within the second set of images that represent the one or more target anatomical structures of the subject body includes detecting a region within an oral cavity of the subject body, on a surface of the subject body, or both within the oral cavity of the subject body and on the surface of the subject body. Feature 3: At least one of (i) detecting the set of image features, (ii) detecting the set of regions, (iii) generating one or more anatomical features, or (iv) generating the predicted medical condition, includes processing one or more items of input data using one or more machine learning models. Feature 4: Processing the one or more items of input data using the one or more machine learning models includes processing a four-channel input tensor to generate the predicted medical condition, where the four-channel input tensor includes a three-channel image of a portion of the subject body and a one-channel anatomical model corresponding to the portion of the subject body. Feature 5: Generating the predicted medical condition includes extracting at least a first value from the anatomical features and at least a second value from the stored medical condition features; and generating the predicted medical condition based on a comparison of the first value and the second value. Feature 6: The comparison of the first value and the second value includes at least one of a distance computation or one or more threshold checks. Feature 7: Actions include generating a treatment recommendation for treating the subject body using the predicted medical condition, where transmitting the signal indicating the generated predicted medical condition to the connected device includes transmitting a signal that indicates the generated treatment recommendation for treating the subject body. Feature 8: Capturing the first set of images includes generating an estimated pose of the subject body within the first set of images; and providing a prompt to indicate an adjustment for the subject body based on the estimated pose. Feature 9: Further including capturing a set of responses to a questionnaire; and generating the predicted medical condition further uses the set of responses to the questionnaire. Feature 10: The set of regions consists of a single region. Feature 11: The second set of images consists of a single image. Feature 12: The set of regions within the second set of images represent at least one of a mandibular region, a malar region, a sub-malar region, a mental region, or a periorbital region. Feature 13: The set of regions within the second set of images represent a jaw of the subject body. Feature 14: The set of regions within the second set of images represent one or more regions of an oral cavity of the subject body. Feature 15: The first set of images is captured using a front-facing camera of a mobile device. Feature 16: Extracting the second set of images includes selecting one or more frames of interest from a video recording of the subject body. Feature 17: Generating the one or more anatomical features includes detecting one or more physical properties of the at least one region, where the one or more physical properties include at least one of a distance measurement, an angle, a volume, or a surface area. Feature 18: The stored medical condition features include data indicative of at least one of obstructive sleep apnea (OSA), a neurodegenerative disease, or a cardiovascular disease. Feature 19: Actions include maintaining a database of (i) the one or more stored feature thresholds and (ii) the one or more stored medical condition features associated with the one or more medical conditions.

The subject matter described in this specification as implemented in particular embodiments realizes one or more of the following technical advantages. Conventional techniques for screening medical conditions like Obstructive Sleep Apnea (OSA) often rely on subjective or processes with accessibility restrictions, such as in-lab sleep studies or cumbersome home tests. The described technologies provide a more efficient, objective, and accessible computer-based solution to screen for the medical conditions.

By programmatically selecting a subset of high-quality images from a larger captured set (e.g., a video stream), the described systems can reduce the computational burden for downstream analysis. This image extraction step, which can filter images based on one or more criteria such as anatomical pose, lighting conditions, or symmetry, can function as a targeted form of data compression or filtering, minimizing redundant processing and storage requirements. By fitting a three-dimensional model to the extracted images and generating specific anatomical features (e.g., relative jaw depth, facial volume ratios, neck circumference), the systems can perform a specialized data transformation that converts generic pixel data into a structured set of clinically relevant metrics. This transformation enables a more accurate and nuanced comparison against stored medical condition features, improving the reliability and precision of the generated medical prediction. The disclosed systems can reduce latency in screening for medical conditions, decrease the number of processing steps required compared to analyzing raw video, and can improve overall accuracy of prediction, e.g., by focusing computational resources on information rich data through pre-processing. Accordingly, the described systems can represent a specific improvement in computer-related technology for medical data analysis. These systems can produce a technical effect of more efficient medical screening, through improved image processing and data analysis techniques, that are faster and/or more cost effective, while being equally or more accurate than conventional techniques.

The described systems can generate a clinical decision support report, e.g., that presents selected high-quality images of the subject alongside graphical overlays indicating key anatomical measurements and identified Obstructive Sleep Apnea (OSA) risk markers. A decision support report can offer a distinct technical improvement. The report can include data visualization techniques to transform analytical results into an intuitive and/or actionable format. This enriched output can improve an efficiency and/or accuracy of a diagnostic process, e.g., by providing overlays or other visual means to enable verification of the system's findings against primary visual evidence.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example medical prediction system.

FIG. 2 shows a system for generating a medical prediction.

FIG. 3 shows a diagram of a number of different regions on the exterior of a subject body.

FIGS. 4A-B shows image segmentation used for processing various oral cavity images.

FIGS. 5A-B show user interface options for a device for predicting one or more medical conditions.

FIG. 6 shows example screens for an operating system to allow for customized interfaces for new or existing patients.

FIGS. 7A-C show a visualization of an example neurodegenerative screening system.

FIG. 8 is a flowchart of an example process for predicting obstructive sleep apnea.

FIG. 9 is a flowchart of an example process for predicting a medical condition.

Like reference numbers and designations in the various drawings indicate like elements.

### DETAILED DESCRIPTION

The technologies described herein generally relate to computer-based systems and methods for screening for medical conditions, such as Obstructive Sleep Apnea (OSA) and its associated comorbidities, neurodegenerative conditions, or cardiovascular diseases, among others. The described systems can capture visual data, such as video scans of a subject's face, neck, and/or oral cavity, often using a readily available electronic device like a smartphone or a tablet computer. From this initial set of visual data, a system as described herein can select a subset of high-quality frames of interest for further analysis. This frame selection process can utilize various techniques, including fiducial feature extraction and head pose estimation, to identify frames that present optimal anatomical views, e.g., filtering out images with potential obstructions, such as dark glasses or facial hair.

Once an optimal set of frames has been selected, the system can perform detailed anatomical analysis. This analysis may involve fitting a two-dimensional (2D) or three-dimensional (3D) model, such as a dense 3D mesh, onto the facial and oral cavity structures depicted in the images. Using these models, the system can identify specific anatomical regions and generate a variety of clinically relevant features. These features can include measurements of relative jaw size and position, assessments of facial symmetry, estimations of neck width, and characterizations of oral cavity structures like the tongue and palate. These generated features can then be processed, for example by deep learning models or rule-based inference systems, to generate a prediction regarding the likelihood of one or more medical conditions that are targeted for diagnosis by the system.

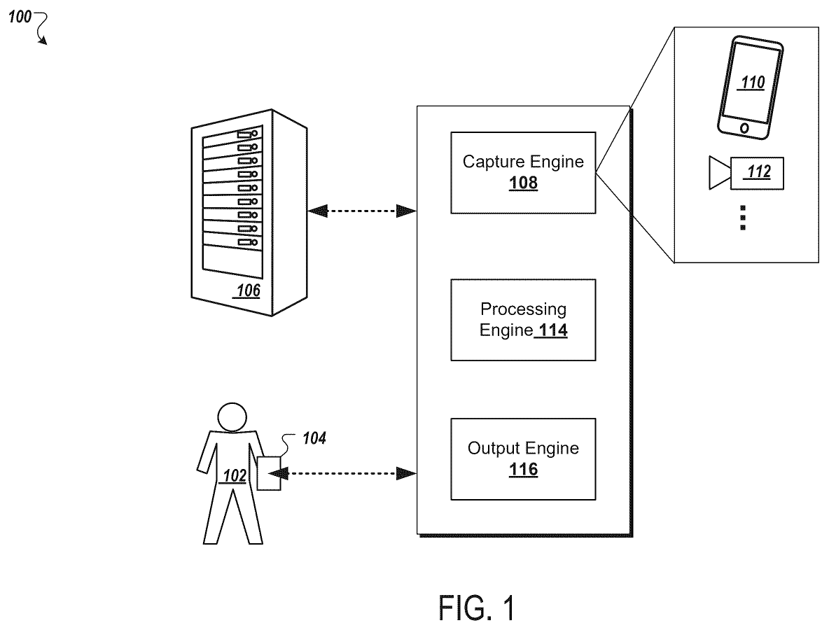

FIG. 1 shows an example medical prediction system 100. The system 100 includes a user device 104 and a backend processor 106. In some cases, processing is performed exclusively on the user device 104 and the backend processor 106 is not used. In some cases, the user device 104 and the backend processor 106 collaborate for processing; each performs at least one operation corresponding to a capture engine 108, a processing engine 114, or an output engine 116.

The user device 104 can be used by a subject body 102. The subject body 102 can be a human or an animal patient. In some implementations, the user device 104 can be used by another entity—e.g., a doctor or other medical professional, or another human assisting the subject body 102. The user device 104 can be a smartphone or tablet computer 110, camera 112, another electronic device with image capture and scanning functionalities, or a combination of these, among other suitable devices.

In some implementations, the system 100 can be used for obstructive sleep apnea (OSA) classification or related conditions. In the following sections, the disclosed technologies are described with respect to the subject body 102 being a human subject. However, as noted previously, the disclosed technologies are applicable to non-human animals as well. Further, in the following sections, the disclosed technologies are described with respect to OSA being a targeted medical condition. However, the disclosed technologies can also be applied to other medical conditions, such as associated comorbidities, neurodegenerative conditions, or cardiovascular diseases, among others for human subjects, or for animal subjects, with the machine learning models being trained to diagnose the various medical conditions based on appropriate training data.

Continuing the description with respect to obstructive sleep apnea (OSA), the system 100 can use images that represent a face, neck, or oral cavity of the subject body 102. In some cases, the system 100 includes a binary classifier—e.g., indicating no obstructive sleep apnea or a prediction that the subject body 102 suffers from obstructive sleep apnea. In some cases, the system 100 includes a multi-class classifier—e.g., no, mild, moderate, or severe obstructive sleep apnea. In some cases, the system 100 detects one or more conditions, such as at least one of mandibular retrognathia, mandibular prognathism, maxillary hypoplasia, micrognathia, submental fullness, deviated septum, macroglossia detection, mallampatti classification, maxillary narrowing detection, malocclusions, malocclusions type 2 and type 3, and/or eye bags.

The system 100 can include one or more trained machine learning models that are configured to detect one or more medical conditions using one or more items of input data, such as images from a camera. For training, runtime, or both, models can process various images, such as one or more images representing a frontal face, profile face, semi-profile face, looking upward face, a tongue sticking out image, an oropharyngeal region, habitual occlusion, or other features of a face of the subject body 102. In some cases, particular types of images are used for prediction of particular medical conditions. For example, images of a fontal face can be used to detect eye bags which can indicate sleeplessness.

The system 100 can capture a first set of images depicting at least a portion of the subject body 102. The first set of images can include one or more images. A capture engine 108, running at least in part on the user device 104, can capture one or more images of the subject body 102. The images can captured by the capture engine 108 can include images of an external portion of the subject body 102, an internal portion of the subject body 102, or a combination of these. In some cases, the system 100 captures images using a front-facing camera of the user device 104, such as a front-facing camera of a smartphone.

In some cases, capturing images can include providing one or more prompts for a user or subject body. For example, the system 100 can generate an estimated pose of the subject body 102 within a first set of images. Using the estimated pose, the system 100 can generate a prompt for the subject body 102 to adjust a pose in some way—e.g., adjusting the pose of the head, oral cavity region, other body parts, or a combination of these. A prompt can be provided visually, e.g., using a display of a device, such as the user device 104. A prompt can be provided in other suitable ways, such as using audio, tactile, or other forms of feedback.

In some cases, the modalities of providing prompts can depend on available interfaces of one or more connected devices. For example, in response to detecting an audio interface, the system 100 can generate an audio prompt that, e.g., describes an adjustment to the subject body 102. In response to detecting a visual interface, such as a display screen, the system 100 can generate a visual prompt that, e.g., shows an adjustment for the subject body 102. In some cases, adjustments improve subsequent processing, e.g., by making input data more similar to training data with which one or more models used for processing have been trained.

In some cases, the capture engine 108 captures one or more modalities of data. For example, the capture engine 108 can capture health data. The health data can be obtained using one or more user devices, such as a smartphones, wearable devices, computers, smart home devices, or a combination of these. Data can include questionnaire data. For example, the capture engine 108 can capture one or more responses to a questionnaire. Subsequent operations, such as operations performed by the processing engine 114 or the output engine 116, can use one or more responses to one or more questionnaires, e.g., for generating a predicted medical condition.

In some cases, the capture engine 108 can detect one or more image features. Image features can include settings used for one or more data captures—such as settings for a camera or other sensor or for processes to obtain data from a user through prompted questions or via scrapping data from user-associated data streams. For example, the capture engine 108 can detect a positioning of the subject body, such as a face positioning. The capture engine 108 can determine whether the positioning satisfies one or more thresholds—e.g., determining if the face is centered or fully visible or a torso is limited to a lower half of an image. The capture engine 108 can determine if a pose of a body part satisfies one or more thresholds—e.g., whether an estimated head pose has a roll, pitch, and yaw within one or more tolerances. The capture engine 108 can determine if an image satisfies an illumination threshold—e.g., whether a detected brightness meets or exceeds a brightness threshold. If the capture engine 108 detects an image does not satisfy one or more image feature thresholds, the capture engine 108 can prompt a subject body, or other user, to adjust a position. If data capture is complete, the processing engine 114 can process one or more image features corresponding to one or more image feature thresholds to extract one or more images from a set of captured images.

Image features can include whether or not a subject body is wearing clothing articles or other paraphernalia that could cause data processing issues, such as glasses, a beard, or scarf. In response to detecting one or more features in an image, the system 100 can perform one or more additional operations. For example, in response to detecting whether the subject body 102 is wearing glasses, the system 100 can determine whether the glasses are dark or not or whether they are occluding a face. In response to subsequent detections, the system 100 can perform subsequent operations. The system 100 can prompt the subject body 102 to remove the worn item that could cause data processing issues, e.g., recommending the subject body 102 removes their glasses for the duration of data capture.

In some cases, in response to determining one or more conditions are satisfied, the system 100 can begin data capture. For example, instead of, or in addition to, capturing data and extracting images for subsequent use in post-processing, the system 100 can ensure one or more initial data capture conditions are satisfied—e.g., that one or more worn items are removed or adjusted to allow for data capture of one or more regions of the subject body 102.

The system 100 can detect a set of image features within one or more images captured by the capture engine 108. For example, the processing engine 114 can detect one or more image features within one or more images captured by the capture engine 108. Image features can include a positioning of a body part or the wearing of an item on the subject body 102.

In some cases, the system 100 extracts one or more images at intervals from a stream of images, e.g., captured as part of video. The system 100 can capture images when a scene changes or at set intervals of time. The system 100 can extract images and run operations on the extracted images. For example, the system 100 can run a pose estimator on the images, such as a head pose estimator.

The system 100 can apply one or more categories to the one or more processed images. For example, the system 100 can categorize an image, e.g., as a frontal, semi-profile, or facing up image. In some cases, the system 100 can categorize an image based on at least one of roll, pitch, or yaw angles. Roll, pitch, or yaw angles can be derived, in some cases, from facial landmarks. Classifications can include at least one of: frontal, profile right/left, slight right/left, facing up, tilt correctable, or do not use. Tilt correction can be applied for one or more images. For images of a head, head tilt correction can be applied. Tilt can be corrected using one or more body landmarks, such as facial landmarks. The system 100 can correct a tilt using landmarks, e.g., based on their X, Y, and Z coordinates and a determined tilt angle.

In some cases, the system 100 can generate an ordered set of images based on one or more image criteria—e.g., a head tilt of the subject body 102. The system 100 can determine whether a least head tilt estimated on a frontal head tilt image satisfies a tilt tolerance threshold. In some cases, in response to determining that the least head tilt image does not satisfy a threshold—e.g., because the head is tilted more than a threshold amount—the system 100 can apply a head tilt corrector. A head tilt corrector can include one or more trained machine learning models or other processes that re-position a body part, such as the head, with a new orientation within an image—e.g., rotating a head portion of the subject body 102 so that the head is not, or less, tilted. In some cases, the system 100 processes only images that either satisfy or have been corrected to satisfy, one or more stored feature thresholds. A stored feature threshold can include a title amount of a head in an image. By processing only a subset of images, such as an extracted subset of images from a larger set of captured images, the system 100 can reduce computational resource usage. The system 100 can reduce a processing time by processing the subset.

The system 100 can access one or more stored feature thresholds. For example, the processing engine 114 can access one or more storage devices, such as a database, to receive one or more feature thresholds. Feature thresholds can indicate features for extracting a subset of images for further processing. Some images may be too dark, incorrectly angled, or obfuscated in some way. The system 100 can improve processing efficiency by extracting a subset of images from a set of captured images. Subsequent processing can then be performed using the subset rather than the entire set of captured images. In this way, computation and energy savings can be achieved.

The system 100 can extract from a set of images captured by the capture engine 108 and using one or more stored feature thresholds a second set of images that satisfy one or more criteria corresponding to the stored feature thresholds. For example, satisfying one or more criteria can include an image orientation being above, below, or within one or more bounds of one or more orientation thresholds. Other features of an image, and other thresholds, can be used for extraction. The second set of images can include one or more images. Extracting can include selecting one or more frames of interest from images, which can represent still images or video. Extracting images can include images of an exterior, interior, or both of the subject body 102.

In some cases, the system 100 can extract one or more selected images. The system 100 can select an image where the subject body 102 is facing front, e.g., towards an imaging device. The selected image can be extracted as part of the subset of images for subsequent processing. In some cases, the system 100 can select an image where the subject body 102 is facing semi-profile right or semi-profile left. In some cases, the system 100 can reflect an image from a left side to appear as an image of a right side of the subject body 102, or vice versa. In some cases, the system 100 can select an image where the subject body 102 is facing up, e.g., where the face of the subject body 102 is point upward from the perspective of an imaging device, such as a camera of the user device 104.

The system 100, upon extracting the second set of images, can detect a set of regions within the second set of images, e.g., that represent one or more target anatomical structures of the subject body 102. The set of regions can include one or more regions.

Anatomical structures can include various regions on the exterior of the subject body 102 or internal to the subject body 102. Anatomical structures of the exterior can include facial structures, such as a nose, eyes, jaw, lips, ears, eyebrows, cheeks, or the like. Anatomical structures of the interior can include oral cavity structures, such as the tongue, teeth, upper mouth, lower mouth, back of the throat, or the like. Detection of regions can be performed by the processing engine 114.

Selected images can be processed—e.g., to generate a two or three-dimensional model, such as a face mesh. A face mesh can include a mapping of one or more facial landmarks onto a captured image. Other meshes can be used for other body parts of the subject body 102, such as arms, legs, torso, oral cavity, among others. Meshes can include suitable landmarks for the body part that are placed according to features identified by the system 100 in captured images—e.g., using one or more trained machine learning models.

In some implementations, the system 100 can detect regions within captured images prior to, or in the absence of, generating a model such as a body part mesh. For example, techniques can be employed to derive a 3D shape of a face from one or more images, such as a video, that captures one or more viewpoints of a body. For example, the system 100 can use Structure from Motion (SfM) and Bundle Adjustment techniques.

The system 100 can extract individual frames from one or more captured images of the subject body 102. On pairs of adjacent frames, the system 100 can identify distinctive features, which can include, e.g., strong edges, corners, or unique textural patterns. These identified features can be matched across one or more pairs of frames. Using these matched feature points, the system 100 can apply triangulation to estimate the 3D position of these points, forming a 3D point cloud of the face.

This process can be repeated for multiple pairs of adjacent frames. Each pair can generate its own 3D point cloud. These individual sparse point clouds can be merged, e.g., by ensuring that any shared points are consistently positioned within a unified 3D coordinate space. The system 100 can perform a bundle adjustment operation, e.g., following the merging of the point clouds. The bundle adjustment operation can refine the 3D positions of the points in the cloud. In some cases, the bundle adjustment operation can refine the positions by optimizing, e.g., concurrently or separately, at least one or more values, such as the 3D point coordinates, camera parameters, or camera poses. The bundle adjustment operation can include optimizing the one or more point clouds. The bundle adjustment operation can return a more accurate and/or cohesive 3D model of the face. The resulting 3D model can be generated without an explicit mesh fitting step. A model can be used to detect anatomical regions or in other analysis or output.

The system 100 can perform various processing operations using a generated model, such as a body part mesh. In some cases, the system 100 can use a generated model to detect one or more regions—e.g., that represent one or more target anatomical structures of the subject body 102. In some cases, system 100 can determine whether or not features represented in the generated model satisfy one or more confidence threshold. In response to one or more feature confidence values not satisfying a confidence threshold, the system 100 can discard a given image and select another image for processing. In some cases, the system 100 can prompt the subject body 102, or another user, to re-capture one or more data items.

The system 100 can generate one or more anatomical features for at least one region of the set of regions that represent the target anatomical structures of the subject body 102. For example, the processing engine 114 can predict one or more distance measurements between various points within a region or between regions. Distance measurements can be one example of anatomical features that represent one or more anatomical structures.

The system 100 can detect one or more regions of the subject body 102. The system 100 can estimate one or more depths of the one or more regions. The system 100 can detect one or more face regions. The system 100 can detect, e.g., a maxilla or mandible regions of a face. The system 100 can use one or more body part landmarks with a generated model of the body to detect specific regions. In some cases, the system 100 maps specific facial landmarks and creates a triangular mesh based on a two or three-dimensional facial model. Regions on a body part can include one or more anatomical structures. For example, regions on a face can cover areas like a nose, cheeks, and chin. Regions can be detected using one or more key points such as the chin (pogonion) or the base of the nose (subnasale). To estimate depth differences, the system 100 can calculate a depth of one or more regions relative to a base region position, such as a nose position. In some cases, a normalizing factor can be used, such as the distance between the eyes. In some cases, the system 100 can compare a median depth of a chin area to a nose base area. The system 100 can use one or more comparisons to generate a predicted medical condition—e.g., comparing a median depth of a chin area to a nose base area can be used to generate a prediction of jaw alignment.

In some cases, the system 100 can generate a region-aware geometric understanding of a face. The system 100 can leverage a two or three-dimensional model, such as a three-dimensional face mesh that is fit onto frontal face images. The system 100 can generate a region-aware geometric understanding of a body part by defining one or more anatomically significant regions of the body part—e.g., based on specific mesh landmark groupings. Regions can include at least one of a mental (chin), mandibular (left/right jawline), malar (inner/mid/lateral cheek), periorbital, glabellar, or temporal areas. Regions can be detected using boundary points from a two or three-dimensional body part model, such as a facial mesh.

The system 100 can compute one or more spatial metrics for a region. The spatial metrics can include at least one of a two-dimensional normalized surface area or a three-dimensional normalized volume. The two-dimensional surface area can be generated by the system through projecting a region's boundary onto an image plane. The system 100 can generate a contour area which can be normalized, e.g., using the total face area. In some cases, the system 100 performs three-dimensional analysis. For example, the system 100 can use depth values from a two or three-dimensional model, such as a face mesh, to build a region-specific model. A region-specific model can include a convex hull. The system 100 can generate a predicted volume of a region-specific model. A predicted volume can be normalized, e.g., by a full facial volume which can be generated by the system 100. The system 100 can detect and quantify subtle morphological deviations, such as micrognathia or facial asymmetries. The system 100 can detect markers for various medical conditions, such as obstructive sleep apnea. To identify key craniofacial markers associated with medical conditions, such as obstructive sleep apnea, the system 100 can be configured to use a multi-view deep learning system. This system may be designed to learn from anatomically relevant sub-regions of an image, such as a face image, to improve prediction accuracy. The learning process can be guided by generating and utilizing a specialized anatomical mask.

In one example implementation, the process can begin by fitting a three-dimensional (3D) model, such as a dense 3D face mesh, onto a two-dimensional (2D) image of a subject's face. This mesh comprises a plurality of vertices, which can be mapped to specific, predefined anatomical regions of the craniofacial complex, such as the mental region (chin), mandibular regions (jawline), malar regions (cheeks), and so on.

Once these anatomical regions are defined by mapping the mesh vertices, the system 100 can generate a single-channel anatomical mask. This mask can be an image of the same dimensions as the input image, where each pixel corresponding to a specific anatomical region can be assigned a unique label or value. For instance, pixels within the mental region could be assigned a first value, pixels within a right mandibular region a second value, and so forth for all defined regions. These values can be scaled to span a desired range, such as the pixel intensity range of a grayscale image. The result is a single-channel image that encodes the spatial location of various anatomical structures.

This generated 1-channel anatomical mask can be used as an additional input channel for a machine learning model, such as a deep learning system. For example, a standard 3-channel RGB image can be combined with the 1-channel mask to create a 4-channel input tensor. By providing this mask, the machine learning model can be guided to focus its learning on specific, anatomically significant regions of the face, potentially enhancing its ability to detect subtle markers associated with conditions like OSA. This approach can allow one or more models to leverage at least one of visual information from the RGB channels and the structural, anatomical context provided by the mask.

A machine learning model of the system 100 can be configured with a dual-branch architecture to process different image views of the subject body 102, for example, a frontal view and a semi-profile view. Each branch of this architecture can be configured to process a 4-channel input tensor. In one implementation, a tensor can include a 3-channel RGB image and a 1-channel anatomical mask. A branch can use a feature extraction backbone, such as, but not limited to, models from the EfficientNet or ResNet families, to process the input and extract relevant visual features.

Features extracted from the respective branches can be fused, for example, through concatenation or another suitable fusion technique, to create a combined feature representation. This fused representation can then be passed to a flexible classification head. The classification head can be adapted for various prediction tasks, such as binary classification (e.g., predicting the presence or absence of a condition), multi-class classification (e.g., predicting the severity of a condition, such as no, mild, moderate, or severe), or multi-label classification (e.g., identifying multiple co-occurring conditions). In some implementations, the model architecture can also include one or more self-attention modules. These modules can be configured to dynamically weigh the importance of different spatial regions within the feature maps, highlighting the most relevant facial or anatomical regions for a given prediction task.

The training process for such a model can be configured to use an appropriate loss function based on the specific classification task. For example, a Binary Cross-Entropy loss function can be used for binary and multi-label classification tasks, while a Categorical Cross-Entropy loss function can be suitable for multi-class classification tasks. To address potential class imbalance within the training data, certain strategies may be employed, such as a two-phase training process. In such a process, initial epochs may use random data sampling, while later epochs may utilize a sampling strategy that enriches batches with samples from underrepresented classes. The performance of the model during training and validation can be tracked using relevant metrics, such as the F1 score or the Area Under the Receiver Operating Characteristic curve (AUROC). The system 100 may be configured to save model checkpoints during the training process, for example, when a performance metric meets or exceeds a predefined threshold, to facilitate efficient retraining or deployment of the best-performing model.

In some cases, a specific deep learning model can be configured to analyze images of an oral cavity. For example, a system can be configured to analyze oral cavity images to identify one or more conditions, such as, but not limited to, Mallampati classification or maxillary narrowing.

To train such an oral cavity analysis model, a dataset of oral cavity images may be collected. In some implementations, the system may apply one or more pre-processing steps, such as applying segmentation masks to the images to isolate and delineate key anatomical regions. These regions can include, for example, the soft palate, the uvula, the pharyngeal wall, or other relevant structures. The system 100 can be configured to perform quality checks on the collected images. For example, the system may analyze images for alignment, image quality, or integrity of the segmentation, and discard images that do not meet certain predefined criteria, such as a vertical symmetry check. Discarding images can be part of extracting one or more images as a second set of images.

Extracted images can be processed to prepare them for model training. Processing steps can include at least one of: resizing the images to a uniform dimension, normalizing pixel values, and applying data augmentation techniques. Augmentation can include operations such as rotation, adjustments to brightness and contrast, cropping, and other transformations to increase the diversity of the training data and improve model robustness. In some implementations, a segmentation mask can be used as an additional input channel for the model, creating a multi-channel input (e.g., a 4-channel image comprising 3 RGB channels and 1 mask channel).

The architecture for such a model can be a single-branch convolutional neural network (CNN). This architecture can use a pre-trained backbone, such as one from the DenseNet or ResNet family of models, for feature extraction. Following the shared backbone, the architecture can include multiple classification heads, with each head configured for a different prediction task. For example, a first classification head can be designed for multi-class Mallampati classification (e.g., Classes I-IV), while a second head can be designed for binary maxillary narrowing detection (e.g., presence or absence). To potentially enhance performance, the architecture may be augmented with additional modules, such as a Spatial Pyramid Pooling (SPP) layer or a self-attention mechanism, which can help the model capture multi-scale features or focus on the most relevant spatial regions within an image.

During the training phase, a combined loss function can be used to optimize the model. For instance, the loss function may be a weighted sum of a Cross-Entropy loss for the multi-class Mallampati classification task and a Binary Cross-Entropy (BCE) loss for the binary maxillary narrowing detection task. To handle potential class imbalance in the training data, techniques such as mini-batch stratification may be employed, which can help ensure that each batch of training data maintains a representative distribution of classes. The performance of the trained model can be evaluated using a variety of metrics appropriate for the specific tasks, such as accuracy for the multi-class classification task or the Area Under the Receiver Operating Characteristic curve (ROC-AUC) for the binary classification task.

In some cases, a machine learning model, such as a deep learning model, of the system 100 can be configured to analyze images of a subject's extended tongue to detect macroglossia, a condition characterized by an abnormally large tongue. For example, a system can collect one or more images where the subject is prompted to extend their tongue out of their mouth. To aid the model, an optional pre-processing step can involve applying a segmentation mask to the images, which can serve to precisely delineate the boundaries of the tongue.

Images in the training dataset can be annotated with binary labels indicating the presence or absence of macroglossia or another medical condition. Before being used for training, these images can undergo pre-processing steps. These steps can include normalization of pixel values to a standard range and various data augmentation techniques to increase dataset diversity and model robustness. Examples of such augmentations can include, but are not limited to, zooming, color adjustments, and minor geometric deformations.

The model architecture for macroglossia detection can be a lightweight, single-branch convolutional neural network (CNN), which can be well-suited for deployment on devices with limited computational resources, such as mobile devices. To leverage learned feature representations, the architecture can utilize pre-trained backbones like DenseNet or MobileNet. In some implementations, a segmentation mask can be provided as input, e.g., as a fourth input channel alongside three RGB channels. This can help a model focus specifically on a morphology of an anatomical region, such as a tongue. The architecture can include additional modules, such as a Spatial Pyramid Pooling (SPP) layer, e.g., to capture shape-sensitive representations at multiple scales.

A model can be trained using a Binary Cross-Entropy loss function, which can be suitable for binary classification tasks. To address potential class imbalance in the training data, where one class (e.g., non-macroglossia) may be more prevalent than another, techniques such as class weighting or adjusted data sampling can be employed. These techniques can help ensure the model does not become biased towards any one particular class, e.g., the majority class. The performance of the model during training and evaluation can be tracked using relevant metrics such as the Area Under the Receiver Operating Characteristic curve (ROC-AUC) or the F1 score. Additionally, model visualization tools like Gradient-weighted Class Activation Mapping (Grad-CAM) can be used to generate heatmaps, which can help verify that the model is focusing its attention on the relevant tongue regions when making a prediction.

In some cases, the system 100 can use one or more image segmentation models to segment one or more images that capture an oral cavity region of the subject body 102. The system 100 can identify one or more images that have an orientation that satisfies one or more orientation thresholds. The stored feature thresholds can include one or more orientation thresholds for extracting one or more images from a set of captured images. In some cases, the one or more orientation thresholds indicate a substantially vertical orientation.

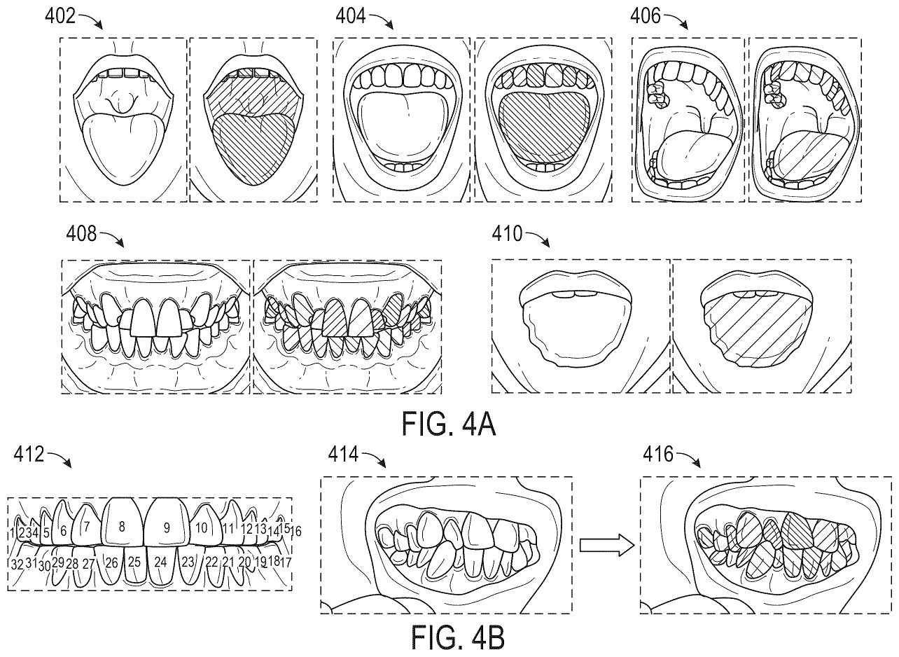

In some cases, the system 100 identifies boundaries of one or more regions of the subject body. For example, the system 100 can identify regions of upper teeth, lower teeth, tongue, or back of the mouth. The system 100 can assess symmetry or physical orientation as part of extracting one or more images for processing. Example oral cavity images are shown in FIGS. 4A-B.

In some cases, the system 100 includes one or more components. The system 100 can include one or more deep learning systems that are trained to generate a prediction for one or more medical conditions. Medical conditions can include sleep apnea, such as obstructive sleep apnea (OSA), central sleep apnea (CSA), or complex sleep apnea syndrome (CompSA). The system 100 can include a clinical decision support system. The clinical decision support system can include a rule-based inference system. The clinical decision support system can estimate a risk score—e.g., a value between 0 and 1—based on one or more detected anatomical feature values. The system 100 can include a decision fusion module. The decision fusion module can receive one or more predicted medical conditions, e.g., generated by one or more learning systems or clinical decision support systems. The decision fusion module can generate a composite risk score for one or more medical conditions, such as a composite score for obstructive sleep apnea.

In some cases, the system 100 generates anatomical features representing estimates of values that describe various features of the subject body 102. In some cases, the system 100 estimates a distance between upper and lower lips, a thickness of lips, and compares one or more values representing the distance and thickness to one or more thresholds. A comparison with one or more thresholds can be used by the system 100 to generate a predicted medical condition—e.g., the subject body 102 may have a dental or skeletal discrepancy that predisposes the subject body to oral breathing. A detected medical condition can be associated with other medical conditions or morbidity factors. Oral breathing can be associated with sleep apnea or other medical conditions.

In some cases, the system 100 estimates at least one of a face width to height ratio, an upper face height to lower face height ratio, a chin height to lower face height ratio, or a combination of these. In some cases, the system 100 estimates a slanted-ness of a nasal bridge, e.g., indicating a potential deviated septum. In some cases, the system 100 identifies one or more symmetrical features—e.g., features on either sides of the face of the subject body 102. The system 100 can measure a difference between features from either side of the subject body 102—e.g., identifying a difference in such features. The system 100 can determine if a detected difference satisfies a disparity threshold, which could indicate facial asymmetry.

In some cases, the system 100 can detect a particular feature of the subject body 102, such as an iris in an eye of the subject body 102. Various features, such as an estimation of margin reflex distance, can be generated using data representing detected aspects of the subject body 102. Using one or more stored medical condition features, such as one or more stored features indicating a likelihood of ptosis or sleeplessness, the system 100 can generate a predicted medical condition.

In some cases, the system 100 detects a certain region corresponding to a portion of the subject body 102, such as a region pertaining to eye bags. The system 100 can run a detector that is trained for the portion of the subject body 102, on that localized portion. For example, the system 100 can run a pre-trained eye-bag detector on a localized region of the subject body 102 that includes an eye-bag portion of the subject body 102. The system 100 can estimate an eye-bag score. The system 100 can use one or more values to estimate a level of sleeplessness.

In some cases, the system 100 generates a model for a portion of the subject body 102, such as an eye-bag region. The system 100 can use a fontal face image and fit a model, such as a face mesh. The system 100 can localize one or more regions on the subject body 102, such as an eye-bag region, which can include an area below the eyes where eye bags typically appear. Regions can be localized, e.g., by identifying one or more face mesh indices indicating a likely location of a given region. The system 100 can crop a portion of a captured image, such as a rectangle or other shape, that includes a localized region on one or more sides of the face. The system 100 can provide images, or a portions of images, to one or more models trained to detect eye bags. The one or more models can include one or more of a visual geometry group (VGG) network, residual network (ResNet), mobile network (MobileNet), efficient network (EfficientNet), or a combination of these among others.

In some cases, the system 100 detects the presence or absence of a given feature. For example, the system 100 can detect if a beard is present or not present. In some cases, a present feature can prevent one or more anatomical features from being generated, such as features that are obfuscated by the presence of a given feature, such as a beard. In some cases, maxilla and mandible depth can be estimated to determine one or more medical conditions, such as retrognathia or mandibular prognathism. In some cases, the system 100 can generate a predicted medical condition of retrognathia in response to detecting that a depth difference between a maxilla and mandible depth satisfies one or more thresholds. In some cases, the system 100 can generate a predicted medical condition of mandibular prognathism in response to detecting that a depth difference between a maxilla and mandible depth satisfies one or more different thresholds—e.g., is less than a value that would indicate retrognathia.

In some cases, the system 100 estimates one or more of the following as part of generating one or more anatomical features for at least one region of the set of regions that represent the target anatomical structures of the subject body: values related to sella-nasion or nasion-points, facial convexity, total facial convexity, nasal angles, chin location, neck width, or nostrils.

The system 100 can access one or more stored medical condition features associated with one or more medical conditions. For example, the processing engine 114 can access one or more memory storage devices that store thresholds indicating one or more medical conditions, such as obstructive sleep apnea. A stored feature associated with a medical condition can include data elements that likely indicate a disease, e.g., a lower jaw length that indicates obstructive sleep apnea.

The system 100 can generate a predicted medical condition, from among the one or more medical conditions, using the anatomical features and the stored medical condition features. For example, the processing engine 114 can compare one or more anatomical features with one or more stored medical condition features associated with one or more medical conditions. Based on one or more comparisons, the processing engine 114 can generate a predicted medical condition. A comparison can include determining whether or not a value satisfies or does not satisfy a threshold value. For example, a comparison that can indicate obstructive sleep apnea can include comparing a lower jaw length with one or more values indicating lower jaw length associated with obstructive sleep apnea and determining that the lower jaw length indicated by the one or more anatomical features is consistent with obstructive sleep apnea.

In some cases, a weighted summation is used to generate a predicted medical condition. For example, the processing engine 114 can process one or more values representing anatomical features of the subject body 102 compared with one or more values representing values that indicate various different diseases. The processing engine 114 might determine that one or more features are above a first threshold but not above a second threshold. Detected feature values can be compared with one or more stored thresholds to determine how the measurements of the subject body 102 compare with stored thresholds. In some cases, a greater number of anatomical structures associated with values linked to a particular medical condition can increase a likelihood of the processing engine 114 generating a prediction that the subject body 102 suffers from that medical condition. In some cases, a number of anatomical structures strongly associated with values linked to a particular medical condition—e.g., by being far lower or higher than one or more thresholds associated with the anatomical structures—can increase a likelihood of the processing engine 114 generating a prediction that the subject body 102 suffers from that medical condition.

In some cases, the processing engine 114 includes one or more machine learning models configured to generate a predicted medical condition. For example, the processing engine 114 can include one or more machine learning models that are trained using data indicating values of anatomical features or other health data—such as questionnaire or life style information. Training data can include a known medical condition associated with a training data set. One or more machine learning models used by the processing engine 114 can be adjusted using feedback from a comparison of predicted output with known medical conditions such that, over time, the models can more accurately predict medical conditions given a set of input data.

The system 100 can transmit a signal indicating the generated predicted medical condition to a connected device. For example, the output engine 116 can transmit a predicted medical condition to the user device 104. The predicted medical condition can include a prediction of obstructive sleep apnea.

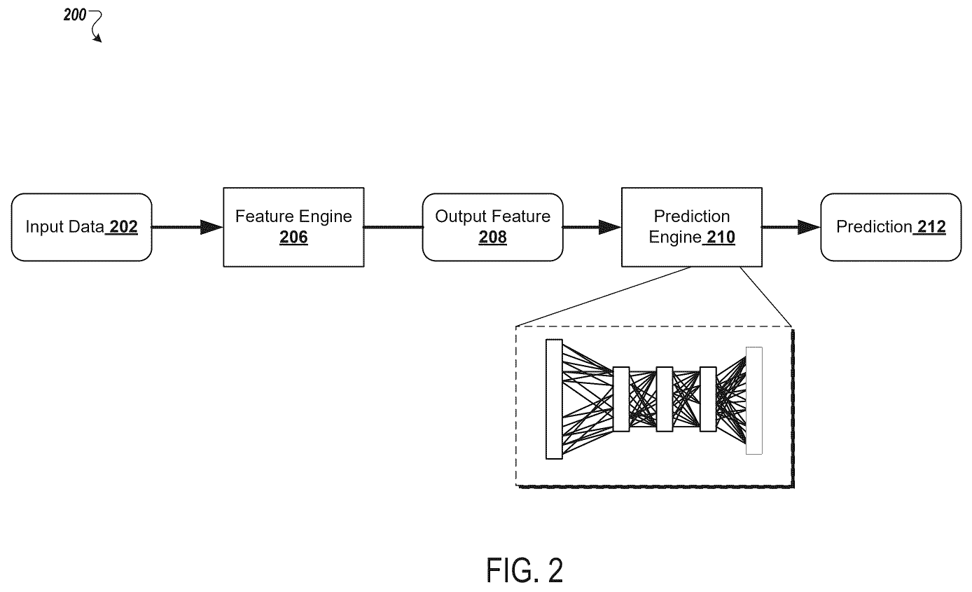

FIG. 2 shows a system 200 for generating a medical prediction. The system 200 includes a feature engine 206 and a prediction engine 210. The prediction engine 210 can include one or more machine learning models, one or more rule-based operations, or a combination of these. The system 200 can be included in the system 100, e.g., operated by the processing engine 114.

The system 200 can obtain input data 202. In some cases, the input data 202 includes images captured of the subject body 102. For example, the input data 202 can include images captured by the capture engine 108 of the user device 104. In some cases, images captured by the capture engine 108 are processed by the feature engine 206. The feature engine 206 can process input data 202 to detect one or more image features 204 within the input data 202.

In some cases, the prediction engine 210 can be configured with multiple data analytics modules. For instance, the prediction engine 210 can include one or more machine learning models, such as one or more deep learning systems, a rule-based inference system, and a decision fusion module. These modules can operate independently or in concert to generate a final medical prediction.

In some cases, the prediction engine 210 includes one or more machine learning systems, such as one or more deep learning systems. A machine learning system can be trained to assess medical conditions, or risks thereof, from various types of input data. For example, a first deep learning system can be trained to perform binary or multi-class classification for obstructive sleep apnea (OSA) using multi-view images of a subject's craniofacial complex, such as frontal, semi-profile, and upward-facing images. A second deep learning system can be trained to analyze images of an extended tongue to detect macroglossia, which can be a strong indicator of OSA. A third deep learning system can be configured to analyze images of the oropharyngeal region to determine a Mallampati classification or to detect maxillary narrowing. Each of these deep learning systems can be configured with a distinct architecture, such as a dual-branch network for multi-view face analysis or a lightweight single-branch network for oral cavity analysis, and can output a probability score, a classification label, or another form of prediction for one or more specific medical conditions.

The prediction engine 210 can include a rule-based clinical decision support system. This system can be configured to operate based on a set of predefined rules and thresholds derived from established clinical knowledge and population data. For example, the rule-based system can receive anatomical features generated by the feature engine 206, such as a neck-width-to-face-width ratio, a mandibular plane angle, or a relative jaw depth difference. The system can compare these measured values against stored reference ranges or thresholds. Based on the degree of deviation of one or more of these measurements from their respective normal ranges, the rule-based system can calculate an individual risk score for a given condition. The rules can be weighted, such that deviations in anatomical features known to be strongly correlated with a medical condition, like a high SNA angle for retrognathia, contribute more significantly to the final risk score.

The prediction engine 210 can include a decision fusion module. This module can be configured to receive and synthesize the outputs from the various other modules, such as the probability scores from the deep learning systems and the risk scores from the rule-based clinical decision support system. The decision fusion module can employ one of several strategies to combine these inputs. For example, it can use a weighted average, where the weights are determined based on the known diagnostic accuracy or confidence level of each input source. In another implementation, the decision fusion module can itself be a trained machine learning model, such as a small neural network or a support vector machine, that has learned to optimally combine the different predictions to produce a more robust and accurate composite risk score. This composite score can represent a holistic assessment of a subject's likelihood of suffering from a medical condition, such as OSA, by leveraging the complementary strengths of both data-driven deep learning analysis and knowledge-based rule-based inference.

In some implementations, a deep learning system can be configured to process images of an oral cavity, such as oropharyngeal imagery and images of a subject's extended tongue. The system can be designed to detect conditions such as Macroglossia, Maxillary narrowing, and to identify a Mallampati class, which are indicators associated with upper airway obstruction and potential obstructive sleep apnea (OSA) risk. The visibility of oropharyngeal structures when a subject's mouth is wide open can serve as an indicator for anatomical obstructions of an airway. In some cases, visibility can be categorized into four classes, such as: Mallampati Class 1, representing full visualization of anatomical structures such as a uvula, tonsils, soft palate, and base of the tongue; Mallampati Class 2, representing partial visualization of a soft palate, tonsils, and the base of the tongue with full visualization of a uvula; Mallampati Class 3, representing partial visualization of a uvula with limited visualization of other anatomical parts; and Mallampati Class 4, representing no visualization of the key anatomical structures. In some systems, a subject identified as Mallampati Class 3 or 4 may be considered at significant risk for OSA. Macroglossia, a condition characterized by an enlarged tongue, can also be a significant indicator for OSA, as the enlarged tongue may partially or wholly block the airway during sleep.

A system, such as the system 100 or the system 200, can be configured to collect oropharyngeal images from a subject in two or more standardized views, such as one with the tongue in a neutral occlusion position and another with the tongue fully extended. For each captured image, the system may generate semantic segmentation masks to delineate key anatomical regions such as the tongue, soft palate, and pharyngeal wall. In some implementations, a state-of-the-art, open-source oral cavity segmentation tool can be used to generate the masks.

The images can then be preprocessed. For example, a system can append a corresponding segmentation mask as an additional input channel to each image, which can create a 4-channel input tensor. As a result, two 4-channel inputs may be created for each subject—one for the tongue-in view and another for the tongue-out view. The system can perform dataset curation by filtering out low-quality images. For example, images can be filtered based on geometric alignment criteria, such as a threshold for vertical symmetry, using the segmentation mask boundaries as a reference. The curated dataset can be partitioned into training and validation subsets, for example, using an 80:20 split, which can help ensure sufficient diversity and class representation in both sets. A system can apply data augmentation techniques, which can include, but are not limited to, random rotation, scaling, color jittering, and mild geometric deformations, to improve model generalization.

The system 200 can be configured with a dual-branch deep learning model. Each branch can be configured to independently process one of the two input image types—tongue-in and tongue-out. A feature extraction backbone, such as, but not limited to, EfficientNet-B7, can be used for feature extraction in each branch. Alternative backbones such as DenseNet, ResNet, or MobileNet can also be evaluated to benchmark performance and resource efficiency for different deployment targets. The features extracted from the two branches can be fused, for example, through concatenation, to form a unified feature representation that captures complementary anatomical cues from both image views.

A fused representation can be provided to two or more distinct classification heads, such as fully convolutional classification heads. For example, a first head can be dedicated to predicting a Mallampati class, which can be an ordinal classification task, and a second head can be dedicated to detecting the presence of Macroglossia, which can be a binary classification task. The model can be trained end-to-end using an optimizer such as the Adam optimizer. A Cross-Entropy loss function can be applied to the Mallampati classification head, while a Binary Cross-Entropy loss can be used for Macroglossia detection. To handle potential class imbalance, mini-batch stratification may optionally be implemented during training to preserve class distribution across batches and reduce bias.

The performance of the model can be evaluated at each training checkpoint using relevant metrics, such as accuracy for Mallampati classification, and ROC-AUC, F1 score, precision, and recall for Macroglossia detection. In some implementations, the system may explore architectural variants by integrating additional modules, such as a Spatial Pyramid Pooling (SPP) layer or self-attention layers, to enhance the model's spatial reasoning capabilities.

In some implementations, to minimize risk and enhance model robustness and reliability across the various deep learning systems described (such as the multi-view face model, the oral cavity model, and the macroglossia model), a structured development and validation process may be adopted. For instance, the process can begin by selecting lightweight model variants as backbones, such as, but not limited to, ResNet-18, EfficientNet-B0, MobileNetV3-small, and DenseNet-121. To leverage learned feature representations and accelerate convergence, these models can be initialized with weights pre-trained on a large-scale, general-purpose dataset such as ImageNet.

Following initialization, the models can be retrained on larger, domain-specific datasets to adapt them to the relevant anatomical structures. For example, a facial analysis model can be retrained on a dataset like Flickr-Faces-HQ, which may comprise approximately 70,000 images, to learn features of facial anatomy. Similarly, an oral cavity analysis model can be retrained on publicly available oral cavity datasets, comprising approximately 6,500 images, to learn features of oral anatomy.

After this domain-specific retraining, the models can be fine-tuned on a specialized dataset, such as a proprietary OSA dataset. This fine-tuning process can be performed incrementally, for instance, by freezing the weights of the earlier layers of the network, which typically learn more generic features, and training only the later, more specialized layers. This approach can help preserve the learned foundational features while adapting the model to the specific nuances of the target dataset.

Throughout the training process, several techniques can be employed to improve training stability and prevent overfitting. Early stopping, based on monitoring validation metrics like loss or F1 score, can be implemented to halt training when performance on the validation set ceases to improve. Intermediate model checkpoints can be saved periodically, which can allow the system to retain fallback options or select the best-performing model from the training run. Generalization can be further improved by using k-fold cross-validation, which helps to reduce training variance by training and evaluating the model on multiple different partitions of the data. Training stability can be maintained through the use of adaptive learning rate schedules, such as learning rate decay or cyclical learning rates, which adjust the learning rate during training.

To ensure that a trained model is focusing on relevant anatomical features when making predictions, visualization techniques like Gradient-weighted Class Activation Mapping (Grad-CAM) or similar methods may be used. These techniques can generate heatmaps that highlight the regions of an input image that were most influential in the model's decision, providing a degree of interpretability. Furthermore, to address any potential class imbalance within the training datasets, techniques such as stratified sampling can be used to ensure that each mini-batch of data includes a representative distribution of each class. Additionally, a weighted loss function, which may combine Binary Cross-Entropy and Focal Loss, can be adopted to give more weight to underrepresented classes during training, which can mitigate bias towards any one particular class.

In some implementations, a machine learning system can be configured to process images of a subject's habitual occlusion, e.g., to identify malocclusion types. Malocclusion, which is a condition describing the misalignment of teeth and jaws, may be an indicator for Obstructive Sleep Apnea (OSA). There may be several classes of malocclusion. For example, a Class 1 malocclusion can be characterized by an upper set of teeth slightly overlapping a lower set of teeth. A Class 2 malocclusion, which can be associated with retrognathism or an overbite, can occur when the upper teeth severely overlap the lower teeth. A Class 3 malocclusion, which can be associated with prognathism or an underbite, can occur when a lower jaw protrudes, causing the lower teeth to severely overlap the upper teeth. Class 2 and Class 3 malocclusions, for example, can be linked to a decrease in oral air space volume, which may increase the risk of OSA. Various visible manifestations of malocclusion can include, but are not limited to, crossbite, overbite, underbite, excessive spacing, overcrowding, abnormal eruption, deep bite, diastema, and overjet.

To analyze these conditions, a machine learning system of the system 200 may be configured to process habitual occlusion images. The input data 202 for such a system can be a multi-channel image. For example, a 4-channel image can be used, which may comprise a 3-channel (e.g., RGB) frontal view of the oral cavity in habitual occlusion, combined with a semantic segmentation mask as a fourth channel. A semantic segmentation mask can be generated, for instance, by an oral cavity segmentation tool, and can highlight anatomical regions relevant to malocclusion, such as teeth, gingiva, and soft tissue outlines.

The model architecture can use a feature extraction backbone, such as, but not limited to, a ResNet-18 architecture, which can be modified to accept the multi-channel input. In some implementations, to enhance a model's ability to capture spatial hierarchies associated with malocclusion, a Global Average Pooling (GAP) layer can be replaced with a Spatial Pyramid Pooling (SPP) layer. The multi-scale features extracted by the SPP layer can be passed through one or more self-attention modules. The self-attention modules can facilitate the modeling of long-range dependencies and contextual interactions between various anatomical regions. The output from the attention layers can then be routed to a classification head, such as a Fully Convolutional Network (FCN) head, designed to classify the malocclusion type and characterize its structural features.

To prepare the data for training, input images may undergo preprocessing, including steps such as alignment, normalization, and data augmentation. Augmentation techniques can include rotation, translation, color jittering, and local elastic deformation. A dataset of such images can be partitioned into training and validation sets, for example, using an 80:20 ratio. In some implementations, data curation can be performed by filtering out samples with poor alignment or low-quality segmentation masks.

The model may be trained end-to-end using an optimizer, such as the Adam optimizer, and an appropriate loss function, such as a categorical cross-entropy loss function. The performance of the trained model can be evaluated using standard metrics, which can include accuracy, precision, recall, F1 score, and confusion matrices. To assess the contribution of specific architectural components, ablation experiments may be conducted. For example, such experiments can be used to compare the performance of a model with SPP and self-attention modules against a baseline architecture, such as a ResNet-18 model with a standard GAP layer. Additionally, interpretability of the model can be assessed using techniques such as Gradient-weighted Class Activation Mapping (Grad-CAM) to verify that the model's attention is focused on anatomically relevant regions when making a prediction.

In some implementations, the system 200 may be configured with a learning model for eye bag detection using frontal face images, which can serve as an indicator of sleeplessness. The process for developing and utilizing such a model can involve several stages. Initially, a frontal face image of a subject body may be obtained, onto which a model, such as a face mesh, can be fit. The system can then localize the regions below the eyes where eye bags typically appear, for instance, by identifying the specific indices of the face mesh corresponding to these areas.

Once localized, a region, such as a rectangular region, that encompasses the identified area on both sides of the face can be cropped from the image. To maintain consistency and comparability in orientation and shape for subsequent processing, the system may keep the cropped image from the right side as-is while performing a mirror reflection on the cropped image from the left side. This operation can be performed on a dataset of frontal face images, which may include hundreds of images with and without eye bags, to create a comprehensive dataset for training, testing, and validating the model.

This dataset can be split into a training set, a test set, and a validation set, for example, using a 70:20:10 ratio. To help ensure data integrity and prevent data leakage, when an image from a particular subject body is assigned to one of the sets, other images of the same subject body can be assigned to the same set. The images, which may initially be in an RGB color format, can be converted to grayscale to reduce computational complexity. The system may also apply data augmentation using various standard techniques to increase the diversity of the training data and improve model robustness.

A lightweight image classification network can be created, using a network architecture as a backbone, such as, but not limited to, a VGG, ResNet, MobileNet, or EfficientNet architecture. To optimize the model for performance and efficiency, some repeated layers in the backbone network may be dropped if the architecture is more than a threshold number of layers. The network can be trained as a two-class classification model using a standard loss function. The training process may be configured to identify a model that meets or exceeds a target performance level, such as, for example, achieving at least 85% precision and 85% recall on the validation set.

Once the system 200 has generated one or more predictions for a given subject body 102, such as an OSA risk probability from a deep learning system and a rule-based risk score from a clinical decision support (CDS) system, the system 200 can be configured to generate a summary report. This report can serve as a clinical decision support tool for healthcare professionals and an informational resource for the subject body 102. The CDS system, in particular, may contribute to this report by visualizing the anatomical measurements and highlighting any significant deviations from established population norms.

In one example implementation, the feature engine 206 can compute a series of craniofacial and oral cavity measurements as described. These measurements can include, but are not limited to, ratios such as face width to face height, angles such as the mandibular plane angle or nasolabial angle, and other metrics like the relative jaw depth difference or neck width. Each of these computed measurements can then be compared by the prediction engine 210 against a database of reference ranges or thresholds, which may be derived from a population of individuals not affected by a particular medical condition, such as OSA.

The CDS system of the prediction engine 210 can analyze these deviations. For instance, the system may quantify the extent to which each measurement deviates from its corresponding reference range. This analysis can form the basis for a rule-based risk score. For example, rules can be defined such that larger deviations in clinically significant features, such as the SNA or SNB angles which relate to jaw position, contribute more heavily to an overall risk score. The final rule-based risk score, which may be a value between 0 and 1, can represent the cumulative risk based on all analyzed anatomical features.

Additionally, the CDS system can generate a visual component for the summary report. This visual report can include one or more images of the subject body 102, such as a frontal or semi-profile view, with graphical overlays that illustrate the key measurements that were taken. For example, lines and arcs can be rendered onto the image to clearly show the angles and distances that were calculated. Measurements that are found to be outside the normal range can be highlighted, for instance, by using a specific color, such as red, to draw the clinician's attention to potential anatomical indicators of risk. The report can also include a table that lists each measurement name, the calculated value for the subject body, the established reference range, and a note indicating any identified craniofacial abnormalities, such as ‘Retrognathic Mandible.’ This combination of quantitative data, visual illustration, and clear identification of abnormalities can provide a physician with a concise yet detailed summary of the anatomical factors contributing to the predicted risk, which can assist in subsequent diagnosis and treatment planning.

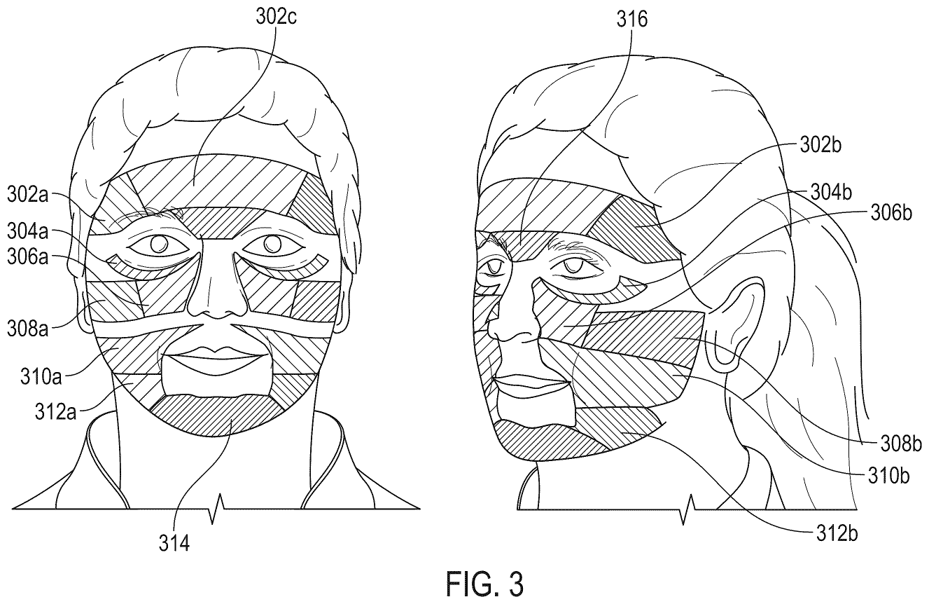

FIG. 3 shows a diagram of a number of different regions on the exterior of a subject body. In this case, the regions cover the surface of the subject body's face and include temporal regions (left, right, and frontal) 302a-c, periorbital regions (left and right) 304a-b, middle-malar regions (left and right) 306a-b, lateral-malar regions (left and right) 308a-b, submalar regions (left and right) 310a-b, mandibular regions (left and right) 312a-b, mental chin region 314, and glabellar region 316. The regions shown in FIG. 3 are for illustrative purposes only and are not limiting. For example, other regions can be detected using the techniques described. Some or all of the regions in FIG. 3, can be detected or none of the regions of FIG. 3 can be detected, in some implementations.

FIGS. 4A-B shows image segmentation used for processing various oral cavity images. Techniques shown in FIGS. 4A-B can be performed by suitable systems, such as the system 100 of FIG. 1 or the system 200 of FIG. 2.

In FIG. 4A, a visualization of image segmentation show how such segmentation can be used to identify a symmetry metric for different regions. A symmetry metric can be used to help assess if a subject's positioning is acceptable for medical condition assessment. In some cases, symmetry metrics can be used as stored feature thresholds, e.g., for use in extracting one or more images from a set of images captured by a capture engine.

A system, such as the system 100 of FIG. 1, can use one or more image segmentation models to segment regions within captured images. Captured images can be filtered using one or more stored feature thresholds—e.g., indicating a correct or expected orientation, such as a vertical orientation. The system 100 can identify one or more boundaries of various regions in an oral cavity, such as upper teeth, lower teeth, tongue, and back of the mouth. The system 100 can assess symmetry or physical orientation in extracting one or more images for further processing.

Images 402, 404, 408, and 410, show a vertical orientation that may be acceptable to an operating system, such as the system 100. For example, the stored feature thresholds used for extracting a second set of images from a first set of captured images can indicate a vertical orientation of the subject body 102 towards an image capture device. The image 406 shows a non-symmetric image that may be excluded from subsequent processing, e.g., included in a first set of captured images but not included in a second set of extracted images.

FIG. 4B shows another example use of image segmentation. In FIG. 4B, teeth crowding can be detected. A teeth number detector can be run on an operating system, such as the system 100. The teeth number detector can include a deep learning model or other suitable machine learning model. The teeth number detector can identify a tooth identifier with each of one or more detected teeth within a captured image. Teeth number identifiers can correspond with the teeth numbers shown in image 412. The system 100 can run image segmentation on an oral cavity image, such as image 414, and generate a segmentation output, visually shown in item 416. Boundaries of one or more teeth can be extracted. The system can determine crowding, e.g., if one or more boundaries of teeth overlap more than a threshold amount. A threshold can be a static value or an aspect of a trained model—e.g., generated as part of a training operation of the corresponding model.

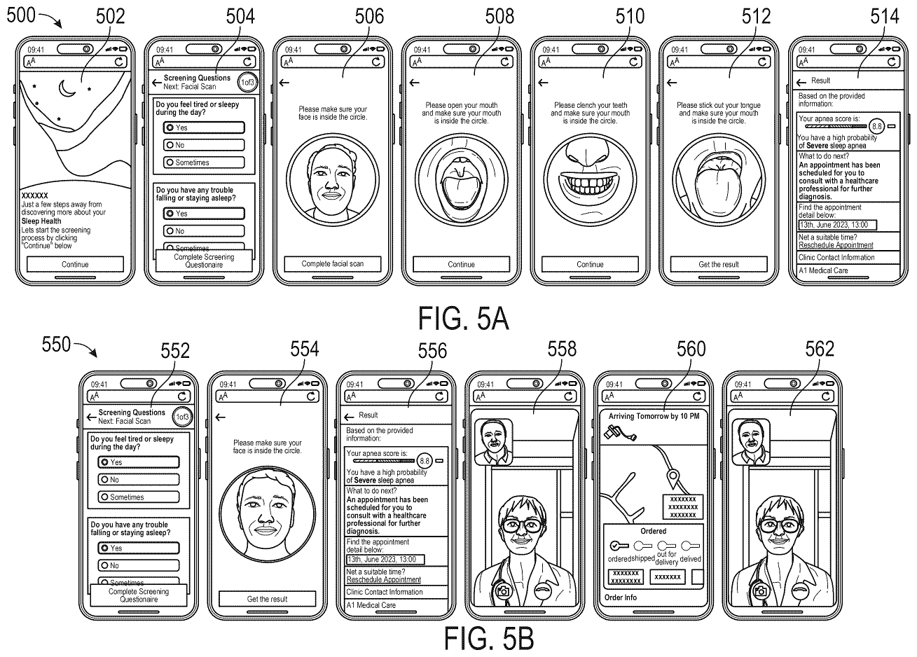

FIGS. 5A-B show user interface options for a device for predicting one or more medical conditions. The device can be the user device 104 of FIG. 1, such as a smartphone. FIG. 5A shows a process 500 that can include one or more of the following screens. For example, the process 500 can include a welcome screen 502 user interface with a selectable option for a user to continue with a prediction process. The process 500 can include a screening questionnaire screen 504—e.g., ‘Do you feel tired or sleepy during the day? Yes/No/Sometimes?’, ‘Do you have any trouble falling or staying asleep? Yes/No/Sometimes?’. Different questions can be used depending on implementation. The process 500 can include a facial scan screen 506. For example, a device can operate a camera or other imaging device to capture an image of a subject body, such as the subject body 102. Images of the subject body 102 can be shown on a display of the device, such as the user device 104. A facial scan can be performed—e.g., generating one or more two or three-dimensional models of a face or other portion of a subject body. The facial scan screen 506 can include capturing prompts, such as ‘Please make sure your face is inside the circle.’ The process 500 can include an interior capture screen 508. The interior capture screen 508 can be used to capture an oral cavity region or other internal region of a subject body. The interior capture screen 508 can include a prompt, such as ‘Please open your mouth and make sure your mouth is inside the circle.’

In some cases, visual indicators, such as colors can show when an image satisfies one or more required image features—such as a red ring changing to a green ring bordering a displayed image or images of a subject body. Non-visual prompts, such as audio or haptic prompts can be generated by a device instead of, or in addition to, displayed visual prompts. In some cases, a prompt can be generated in another modality in response to a detection of a subject body. For example, if a system, such as the system 100, detects that the subject body 102 is not adjusting a pose of a body portion as directed using a first modality of a prompt, the system 100 can use a second modality, e.g., providing an audio prompt after the system 100 detects that the subject body 102 does not adjust position after display of a textual prompt on a display screen.

The process 500 can include a body operation capture screen 510. In this case, the body operation is clenching teeth. A prompt of ‘Please clench your teeth and make sure your mouth is inside the circle’ can be provided. Other operations can be prompted by a system, such as the system 100, as appropriate. Capturing operations performed by the body can aid in medical condition prediction as it shows activations of various anatomical features that can aid in determining, or ruling out, various medical conditions or severity of one or more conditions. The process 500 can include a second body operation capture screen 512. The second operation includes sticking the tongue out. The subject body 102 can be prompted to stick their tongue out for imaging—e.g., using a textual prompt, such as ‘Please stick out your tongue and make sure your mouth is inside the circle.’ The process 500 can include presenting a result screen 514. The result screen 514 can include a risk score, such as a score from 0 to 10 or 0 to 100, or any other suitable numerical, alphanumerical, color-based, or other indicative scale. An operating system can determine whether to automatically schedule an appointment for follow-up or suggest the option of an appointment to a user, which may be the same as the subject body or may be different. In some cases, severity of a medical condition that satisfies a threshold can cause the system 100 to automatically schedule an appointment. A user can then cancel or adjust the scheduled appointment as needed. Auto-scheduling can help ensure that individuals that are higher risk are seen by appropriate specialists more quickly—e.g., without a lag between getting medical predictions and manually scheduling appointments. For medical conditions in which days or hours can mean the difference between life and death, auto-scheduling appointments using medical condition predictive thresholds can save lives.

FIG. 5B shows a process 550 that can include one or more of the following screens. The screens, like the process 500, can be displayed on one or more operating devices, such as the smartphone 110 operating as the user device 104 of FIG. 1.

The process 550 can include a questionnaire screen 552. The process 550 can include obtaining biometric data, e.g., from one or more connected devices, such as wearable devices. The process 550 can include a data processing screen 554. For example, an image captured of a subject body can be displayed and textual or other prompts can be provided to a user, which can include or be different from the subject body. Prompts can include prompts for data capture or status of processing. The process 550 can include a result screen 556. The result screen 556 can include a displayed indication of a predicted medical condition, such as a numerical, alphanumeric, or other type of score. The result screen 556 can indicate one or more scheduled appointments. Appointments can be setup manually or automatically by an operating system, e.g., that operates the process 550. The process 550 can include a telehealth screen 558. The telehealth screen 558 can include text or video chat features to allow communication between a medical professional and a subject body, which can be a patient. The process 550 can include a shipping status screen 560. The shipping status screen 560 can show various medical equipment related to a predicted medical condition, or equipment suggested based on a telehealth visit, that was ordered and is being delivered to the home of a subject body. The process 550 can include additional therapy or follow-up telehealth appointments. In some cases, data captured over time can be used to track a medical condition over time. If a medical condition worsens or improves, an operating system can perform operations in response. Such operations can include scheduling or canceling telehealth visits, ordering or canceling medical equipment, or a combination of these among others.

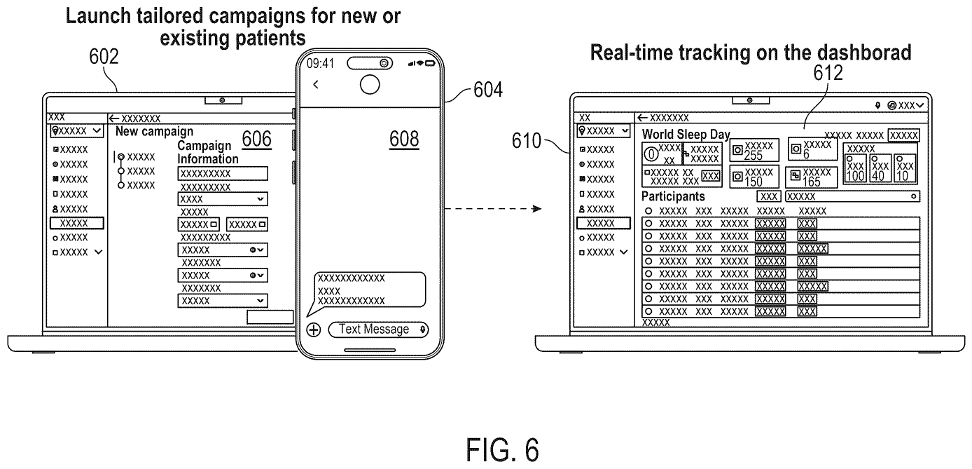

FIG. 6 shows example screens for an operating system to allow for customized interfaces for new or existing patients. Interfaces can be customized to capture specific data for specific medical conditions. The data can be processed differently for different interfaces. For example, an interface can be generated for sleep apnea. The interface can include generating screens for capturing specific type of data that is predictive of sleep apnea or other related conditions. The data captured can be processed using one or more trained machine learning models or rules-based architectures. These processes can be fine-tuned for the particular medical condition, such as sleep apnea or other medical condition. Results for users can be tracked. In some cases, longitudinal data can be used to assess recommended treatments or condition severity, among other medical features.

The screens shown in FIG. 6 can be part of a configurable platform, which can be referred to as an experience studio, designed to allow administrators, such as clinicians or telehealth providers, to create, customize, and manage various user journeys and clinical workflows. Such a platform can facilitate flexible deployment and configuration of the medical prediction technology described herein. A first device 602, which can be any suitable personal computer, can display a first screen 606. This first screen 606 can represent a customization interface within the platform for setting up a new campaign or workflow. This interface can provide various settings and options for configuring the campaign, such as defining the sequence of data capture operations. For example, an administrator could use this screen to select specific data capture modules, like a facial scan module or an oral cavity video module, and specify the order in which they are presented to a user. The first screen 606 can also allow for the configuration of data processing pipelines, enabling the administrator to specify which machine learning models or rule-based inference systems should be invoked for the data obtained during the campaign.

In some implementations, the configuration of a campaign can be facilitated by an intelligent assistant. A second device 604, which can be a smartphone or another computer, can display a second screen 608. The second screen 608 can provide a user interface with a machine learning assistant configured to assist in the setup of one or more campaigns. For example, this assistant could provide recommendations on which data capture modules are most relevant for screening a particular medical condition, or it could suggest optimal parameters for a machine learning model based on the target demographic of the campaign. This can streamline the process of creating effective and clinically relevant screening workflows, e.g., for administrators without deep technical expertise in machine learning or data science. The assistant could be interactive, allowing a user to specify goals (e.g., “screen for pediatric OSA”) and then generating a draft workflow configuration in response.

Once a campaign is launched, its progress can be monitored through a dashboard. A third device 610, which can be the same as or different from (e.g., another computer) the first device 602, can display a third screen 612. The third screen 612 can indicate a dashboard providing a view of an active campaign. This dashboard can be used by various personnel, such as a subject, a patient, a doctor, or a campaign administrator, to track data collection and analysis. The dashboard can display data for various participants, including their associated data, their current status in the workflow (e.g., “questionnaire complete,” “facial scan pending”), or their progress through the screening process. This allows for the tracking of data as it flows through the configured system, from initial capture to final analysis, providing insights into engagement rates, completion times, and preliminary results, thereby enabling effective management and oversight of the screening process.

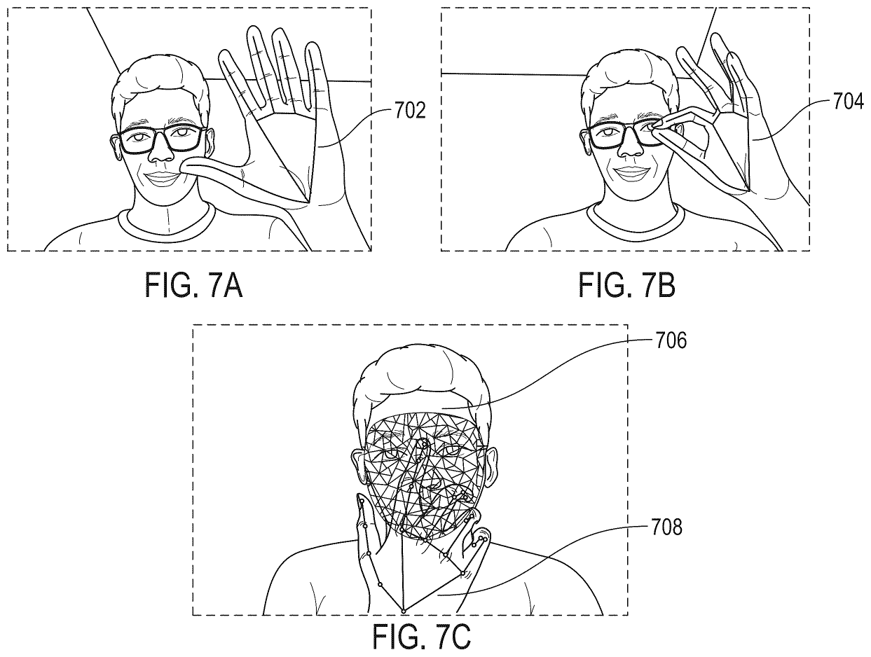

FIGS. 7A-C show a visualization of an example neurodegenerative screening system. The system can assess differences in finger tap rate between left hand and right hands. The neurodegenerative screening system can be implemented on the system 100 or other suitable system. Although described for neurodegenerative screening, the techniques shown and described in regard to FIGS. 7A-C can be used for screening one or more other medical conditions. Different medical conditions can be screened for using one or more customized interfaces, e.g., as shown in FIG. 6. An administrative user can identify parameters for an interface that include at least one of: one or more types of data to capture, prompts or acceptable data, and processing techniques of that data. Identified parameters can change depending on a medical condition to be identified or a given administrative user as different administrative users may use different techniques for various diagnostics.

In FIG. 7A, a subject body is shown being prompted by a system, such as the system 100, to face the camera and show their left hand with the palm facing the camera, fingers spread apart and hand steady, e.g., ensuring their wrist and fingers and a portion of their forearm are visible to the camera.

In FIG. 7B, the subject body is asked to touch the tip of their index finger to the tip of their thumb to form a closed position (like an OK sign) and subsequently release the index finger back to its original position, keeping the palm facing the camera. In some cases, the subject body can be prompted to repeat this open-close motion (index finger touching thumb, then releasing) multiple times (e.g., 15 times) in a smooth and controlled manner. Operations performed on the left hand can be repeated for the right hand. The order of hands can be adjusted, depending on implementation.

One or more images of the movement of the hand can be captured, e.g., as part of the data capturing performed by the data capture engine 108. The processing engine 114 can extract one or more images from the captured images. The processing engine 114 can fit a hand mesh onto the hand—as shown in items 702 and 704. The processing engine 114 can identify one or more indices indicating a tip of an index finger and a tip of the thumb finger. This can be included in an operation of detecting a set of regions within a set of images extracted from a first set of images that represent one or more target anatomical structures of the subject body. In this case, the anatomical structures include a hand structure.

The processing engine 114 can compute a distance between the index finger and the thumb finger—e.g., as part of generating one or more anatomical features for at least one region of the set of regions that represent the target anatomical structures of the subject body. The processing engine 114 can identify a threshold indicating whether or not the index finger and the thumb finger were in closed or open form. If multiple adjacent images indicate ‘closed form’, the processing engine 114 can apply a filter to identify those images as a single ‘closed form’ event. The processing engine 114 can estimate one or more metrics. For example, the processing engine 114 can estimate a time taken by subject to perform ‘closed form’ event on the right hand and a time taken for the same on the left hand. The estimates can be used with one or more accessed medical condition features associated with one or more medical conditions to generate a predicted medical condition. For example, a medical condition feature can indicate a neurodegenerative medical condition when a time amount of a hand, or a time difference between the hands, satisfies one or more thresholds. The processing engine 114 can use a comparison of estimated values with one or more thresholds o determine one or more predicted medical conditions.

FIG. 7C shows another example data capture process—e.g., for glabellar tap reflex. In the example of FIG. 7C, the subject body is prompted by an operating system, such as the user device 104, to tap their forehead using their index finger (left or right) at a moderate pace, e.g., one tap every few seconds. Data capture can conclude when the data capture engine 108 determines that a subject body has provided a sufficient amount of data, e.g., images that represent a threshold number of taps, such as ten.

The processing engine 114 can extract one or more images from a captured set of images. The processing engine 114 can generate a two or three-dimensional model that fits the face and hand. The processing engine 114 can use blink detection to identify time-stamps when the subject blinked. The processing engine 114 can track the tip of the index finger. The processing engine 114 can identify one or more images, or timestamps, when the tip of the index finger touched the forehead. In some cases, depth values of detected fingers or forehead can be used as thresholds for determining location of forehead and finger and corresponding tapping events. The processing engine 114 can use filters to count a tap event once, not repetitively for the same tap event. The processing engine 114 can determine a time between a tap-event and a blink event. In some cases, if the subject has a tendency to blink every time they tapped their forehead or otherwise, the processing engine 114 can generate one or more values that represent the reflex. The processing engine 114 can use the one or more values along with one or more stored medical condition features associated with one or more medical conditions to generate one or more predicted medical conditions.

In some implementations, a system such as the system 100 or the system 200, can be configured to screen for medical conditions that are comorbid with a given medical condition, such as OSA. The system can leverage the same or similar data capture and analysis infrastructure. The engines of the system can perform various operations to detect markers associated with neurodegenerative disorders, cardiovascular conditions, stroke, and other systemic health complications linked to OSA. This can be achieved through dedicated processing pipelines that analyze specific biometric data captured from the subject body 102, e.g., using at least one of machine learning models or rule-based inference systems, which can be operated by the prediction engine 210.

For example, to screen for neurodegenerative conditions like Parkinson's disease, the system can guide a subject body 102 through a series of specific motor tasks captured via video by the capture engine 108. One such task can involve assessing differences in finger tap rates between the left and right hands. As illustrated in FIG. 7A and FIG. 7B, a subject can be prompted to repeatedly touch the tip of their index finger to the tip of their thumb for a set duration, first with one hand and then the other. The processing engine 114 can process the captured video, applying a hand mesh model (e.g., items 702, 704) to the extracted frames to track the positions of the fingertips. As part of generating anatomical features, the processing engine 114 can compute the distance between the index finger and thumb tips in each frame, identify ‘open’ and ‘closed’ events based on a distance threshold, and calculate the frequency and consistency of these taps for each hand. The prediction engine 210 can then access stored medical condition features—such as normative tap rate thresholds or expected symmetry between hands—to generate a prediction. A significant disparity in tap frequency or a rate below a certain threshold can indicate bradykinesia, a potential marker for a neurodegenerative condition.

Another operation for neurodegenerative screening involves assessing the glabellar tap reflex, as depicted in FIG. 7C. The capture engine 108 can record video of the subject tapping their forehead. The processing engine 114 can analyze this video by fitting both a face mesh 706 and a hand mesh 708 to the images. It can then detect ‘tap events’ by identifying images where the distance between the fingertip and the forehead falls below a certain threshold. Concurrently, the processing engine 114 can detect ‘blink events’ by monitoring the distance between the upper and lower eyelids. The prediction engine 210 can then analyze the temporal correlation between tap events and blink events. A persistent blink reflex in response to repeated tapping (Myerson's sign) can be a stored medical condition feature indicative of frontal release signs associated with certain neurodegenerative disorders. The output engine 116 can then transmit a signal indicating a prediction based on this analysis.

The system 200 can be configured to detect signs of cardiovascular conditions or stroke, which can be comorbidities of medical conditions, such as OSA. For instance, the feature engine 206 can analyze the RGB color values of pixels within different facial regions identified by a face mesh to detect cyanosis (a bluish discoloration of the skin indicating poor oxygen circulation) or xanthalesma (yellowish bumps around the eyes, which can be linked to high cholesterol). The prediction engine 210 can compare these detected color patterns against stored features representing normal skin tones. Similarly, to detect signs of a past stroke, the feature engine 206 can assess facial symmetry using fiducial landmarks from a face mesh. Asymmetry, such as a drooping on one side of the mouth or eye, can be quantified and compared by the prediction engine 210 against a symmetry threshold. A significant deviation can be flagged as a potential indicator. In another operation, the feature engine 206 can be configured to detect Frank's sign, a diagonal crease in the earlobe associated with an increased risk of coronary artery disease, by running an edge detection algorithm on a localized image of the ear. The presence and prominence of such a crease can be used by the prediction engine 210 to contribute to an overall cardiovascular risk assessment. These various detection operations can be performed by at least one of a machine learning model, e.g., trained on labeled images, or by rule-based systems. Detection operations can be performed by the systems described herein, such as the system 200 and the prediction engine 210.

A system, such as the system 100, can incorporate or be deployed via a configurable platform, which may be referred to as an experience studio. This platform can be designed to allow administrators, such as clinicians or telehealth providers, to create and manage customized user journeys and clinical workflows using low-code or no-code tools. Such a platform can facilitate the flexible deployment and configuration of the medical prediction technology, which can be a distinct system-level functionality separate from the specific screening methods themselves.

The platform can be configured as a multi-tenant, cloud-based system that allows for the configuration of different ‘products’ or ‘campaigns’ based on the same core technology. For example, a provider can configure a screening-only product for a public awareness campaign, where the subject body 102 receives a direct risk score and a suggestion to schedule an appointment. In another configuration, the provider can create a clinical decision support (CDS) workflow. In this CDS workflow, the subject body 102 may go through the same data capture process, but the results and detailed anatomical analyses are automatically integrated into a connected Electronic Health Record (EHR) system (e.g., Athena Health™), for review by a physician, without necessarily being shown directly to the subject.

To create these customized journeys, the platform can include a graphical user interface-based workflow editor, or ‘experience studio.’ Within this studio, an administrator can define a sequence of user interactions by selecting and arranging pre-built modules or ‘plugins.’ These modules can correspond to various operations, such as displaying a welcome screen, presenting a specific questionnaire, initiating a facial scan, requesting an oral cavity image, or calling a specific analytical algorithm. The administrator can configure the logic and content for each module, for example, by defining the specific questions in a questionnaire, setting the text for user prompts (e.g., ‘Get ready for face scan’), and specifying the branding and visual theme for the user interface.

The platform's orchestration engine can execute these defined workflows. When a user journey is initiated, for example via a QR code, an API call, or an SDK embedded in a third-party application, the orchestration engine can step through the configured sequence. It can dynamically determine which data capture mechanisms to activate and in what order. For instance, a workflow could be configured to first ask a set of questions and, based on the responses, decide whether to proceed with a facial scan, an oral cavity scan, or both.

After data is captured, the workflow can be configured to call the appropriate analysis algorithm or model. The platform can maintain a registry of different analytical modules, each tailored for a specific condition (e.g., OSA or a neurodegenerative disorder) or data type (e.g., facial video, oral cavity images, wearable data). The workflow definition can specify which algorithm to invoke based on the campaign, the captured data, or other contextual information. For example, a workflow for a sleep clinic might call an OSA prediction model, while a workflow for a neurology practice might call a model designed to detect signs of a targeted neurodegenerative disorder, such as Parkinson's disease. In this manner, the system can support a multi-modal and multi-condition analysis capability, all configurable through the same platform. This allows providers to rapidly deploy and iterate on different screening and patient intake solutions without requiring custom software development for each use case.

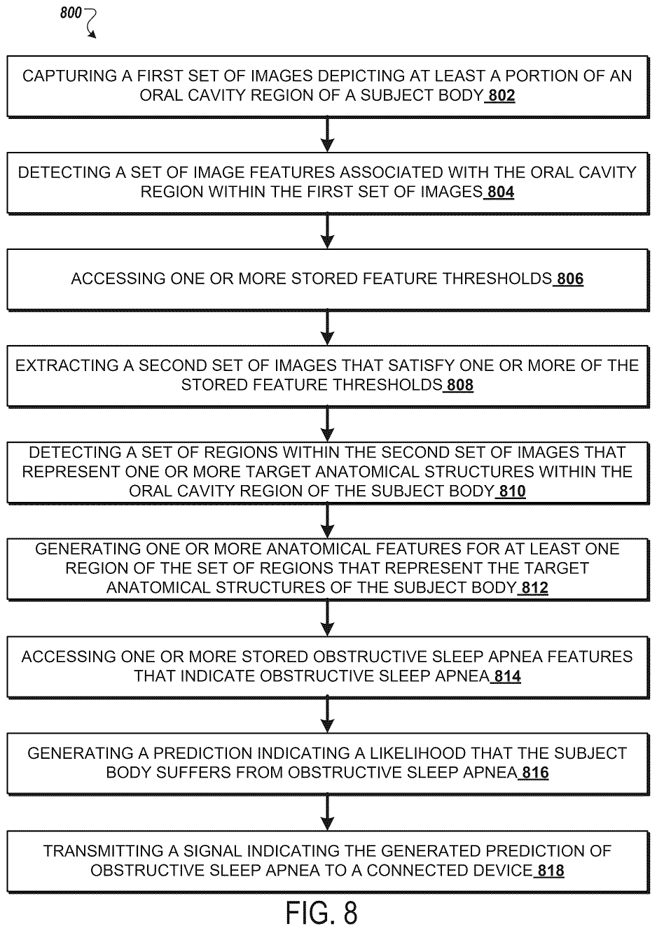

FIG. 8 is a flowchart of an example process 800 for screening for obstructive sleep apnea. For convenience, the process 800 will be described as being performed by a system of one or more computers, located in one or more locations, and programmed appropriately in accordance with this specification. For example, a medical prediction system, e.g., the medical prediction system 100 of FIG. 1, appropriately programmed, can perform the process 800.

The process 800 includes capturing a first set of images depicting at least a portion of an oral cavity region of a subject body (802). For example, the capture engine 108, operating on a user device 104 such as a smartphone 110, can capture video of the subject body 102 opening their mouth as prompted by a user interface, such as the interface shown in the interior capture screen 508.

In some cases, capturing the first set of images can be performed using a handheld image capture device, such as a smartphone. For example, the capture engine 108 of the user device 104 can utilize a built-in camera, such as a front-facing or rear-facing camera of a smartphone 110, to record the video of the oral cavity region. A user can hold the smartphone and position it according to on-screen prompts to ensure the oral cavity is properly framed. In other examples, a clinician can use the handheld device to capture images from the subject body 102.

In some implementations, capturing the first set of images can also include capturing an image of a surface region of a head of the subject body. For example, in addition to the oral cavity images, the capture engine 108 can capture images of the subject's face from various angles, as depicted in the facial scan screen 506. The process can then involve detecting a second set of image features associated with the head, such as fiducial landmarks. The processing engine 114 can then access stored feature thresholds corresponding to both oral cavity features (e.g., symmetry) and head features (e.g., specific head poses). Using these thresholds, the processing engine 114 can extract a first image that satisfies an oral cavity threshold and a second image that satisfies a head feature threshold, creating a combined set of images for analysis.

The process 800 includes detecting a set of image features associated with the oral cavity region within the first set of images (804). For example, the processing engine 114 can apply an image segmentation model to each frame of the captured video. The segmentation model can identify distinct areas, allowing the processing engine 114 to detect subregions such as an upper teeth region, a lower teeth region, a back of the mouth region, or a tongue region, as shown in FIGS. 4A-B. The presence and clarity of these regions can serve as the detected image features.

In some implementations, the system can first generate a three-dimensional model of at least a portion of the subject body, such as the head or oral cavity. The detection of regions within the extracted images can then be performed using this three-dimensional model. For example, the processing engine 114 can fit a dense 3D mesh to the extracted images, and specific vertices or groups of vertices on the mesh can be pre-mapped to correspond to target anatomical structures. For instance, the system can detect a lower jaw region and an upper jaw region by identifying the group of mesh vertices that define these respective anatomical areas on the generated three-dimensional model.

The process 800 includes accessing one or more stored feature thresholds (806). For example, the processing engine 114 can query a database that stores criteria for high-quality images suitable for analysis. These thresholds can include an axis of symmetry feature threshold, which specifies a required degree of vertical alignment for the segmented regions (e.g., upper teeth, tongue) within an image, as illustrated by the acceptable images 402, 404, 408, and 410 in FIG. 4A.

In some cases, accessing the one or more stored feature thresholds can involve accessing an axis of symmetry feature threshold that indicates a required degree of symmetry for one or more subregions of the oral cavity. For instance, the processing engine 114 can retrieve a threshold parameter specifying that the vertical axes of symmetry for the upper teeth region, lower teeth region, and tongue region are required to be aligned within a certain percentage. Extracting the second set of images would then involve selecting only those images where the detected subregions meet this vertical alignment criterion, ensuring a frontal and properly oriented view.

The process 800 includes extracting a second set of images that satisfy one or more of the stored feature thresholds, e.g., from the first set of images and using the stored feature thresholds (808). For example, the processing engine 114 can filter the video frames, selecting only those frames where the segmented oral cavity regions are well-defined and exhibit a vertical symmetry that meets the stored threshold. This operation can reject misaligned frames like image 406 from FIG. 4A, effectively compressing the video data into a smaller, higher-quality subset of images for subsequent processing.

The process 800 includes, upon extracting the second set of images, detecting a set of regions within the second set of images that represent one or more target anatomical structures within the oral cavity region of the subject body (810). For example, after selecting the high-quality frames, the feature engine 206 can use the existing segmentation masks to precisely delineate the boundaries of target structures like the soft palate, the uvula, and the pharyngeal wall within those frames.

The process 800 includes generating one or more anatomical features for at least one region of the set of regions that represent the target anatomical structures of the subject body (812). For example, using the delineated regions, the prediction engine 210 can calculate a Mallampati classification score based on the visibility of the soft palate and uvula, or it can measure the relative size of the tongue to detect macroglossia. These scores and measurements constitute the generated anatomical features.

The process 800 includes accessing one or more stored obstructive sleep apnea features that indicate obstructive sleep apnea (814). For example, the prediction engine 210 can access a database storing clinical data, such as rules linking specific Mallampati scores (e.g., Class 3 or 4) or tongue sizes to a high probability of OSA.

The process 800 includes generating a prediction indicating a likelihood that the subject body suffers from obstructive sleep apnea, e.g., using (i) the anatomical features for the at least one region of the oral cavity of the subject body and (ii) the stored obstructive sleep apnea features (816). For example, the prediction engine 210 can compare the calculated Mallampati score and macroglossia measurement for the subject body 102 against the stored OSA features. If the subject's features match the stored indicators (e.g., a Mallampati Class 4 score), the prediction engine 210 generates a prediction indicating a high likelihood of OSA.

In some implementations, the system can be configured to screen for medical conditions comorbid with obstructive sleep apnea. For example, a system can screen for a neurodegenerative condition by capturing a second set of images depicting a motor task performed by a hand, such as repeatedly touching the index finger to the thumb. The processing engine 114 can detect hand features, such as fingertip locations, from these images and generate movement metrics, like the frequency of contact. The prediction engine 210 can then use these metrics to generate a prediction about the likelihood of a neurodegenerative condition.

In some cases, a system can screen for a cardiovascular condition by detecting a region corresponding to an earlobe within the captured images. The feature engine 206 can analyze this region to detect the presence of a diagonal crease (Frank's sign). In response to detecting this crease, the prediction engine 210 can generate a prediction indicating an increased risk of coronary artery disease.

In some cases, a system can screen for a potential past stroke. The feature engine 206 can identify multiple fiducial landmarks on the subject's face within the captured images. The processing engine 114 can calculate a degree of facial symmetry using these landmarks. If the degree of symmetry is below a predefined threshold, the prediction engine 210 can generate a prediction indicating a likelihood of a past stroke.

The process 800 includes transmitting a signal indicating the generated prediction of obstructive sleep apnea to a connected device (818). For example, the output engine 116 can send the generated prediction to the user device 104, where it is displayed to the user via an interface, such as the result screen 514 or the result screen 556. In some cases, transmitting the signal includes presenting, on a display device, information indicating the generated prediction of obstructive sleep apnea or other medical condition.

The transmission of the signal indicating the generated prediction of obstructive sleep apnea can comprise presenting the information on a display device. For example, the output engine 116 can generate a user interface, such as the result screen 514 or result screen 556, that visually displays the prediction to the subject body 102 or another user, such as a clinician, via the user device 104. This presentation can take various forms, including a numerical risk score (e.g., a value from 0 to 100), a categorical classification (e.g., “low risk,” “moderate risk,” “high risk”), or a graphical representation. The user interface can also include explanatory text, visualizations of the anatomical features that contributed to the prediction, and actionable next steps, such as an option to schedule a consultation or connect with a telehealth provider. This allows for the direct communication of the screening result in an accessible and understandable format.

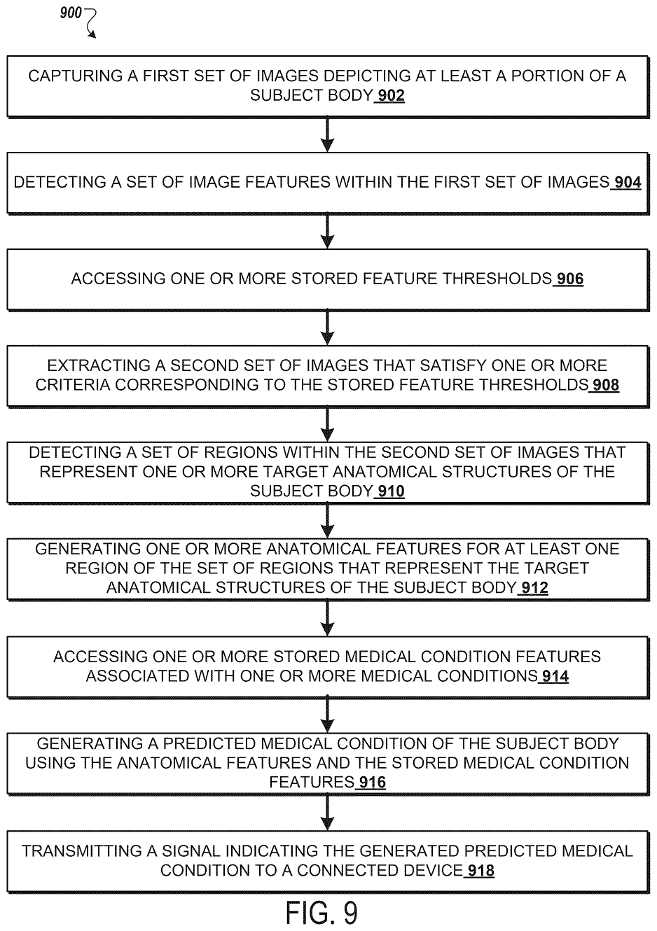

FIG. 9 is a flowchart of an example process 900 for predicting a medical condition. For convenience, the process 900 will be described as being performed by a system of one or more computers, located in one or more locations, and programmed appropriately in accordance with this specification. For example, a medical prediction system, e.g., the medical prediction system 100 of FIG. 1, appropriately programmed, can perform the process 900.

The process 900 includes capturing a first set of images depicting at least a portion of a subject body (902). For example, the capture engine 108, operating on the user device 104, can capture a video of the subject body 102 as the subject follows on-screen prompts, such as those shown in FIG. 5A.

The process 900 includes detecting a set of image features within the first set of images (904). For example, the processing engine 114 can execute a head pose estimator on individual frames of the captured video to determine roll, pitch, and yaw angles, which serve as the image features.

The process 900 includes accessing one or more stored feature thresholds (906). For example, the processing engine 114 can access a database that stores predefined thresholds for image quality, such as acceptable ranges for head pose angles (e.g., yaw angle between 10 and 45 degrees for a semi-profile view) or minimum illumination levels.

The process 900 includes extracting, e.g., from the first set of images and using the stored feature thresholds, a second set of images that satisfy one or more criteria corresponding to the stored feature thresholds (908). For example, the processing engine 114 can analyze the video captured by the capture engine 108 and select only the frames where the estimated head pose angles fall within the stored thresholds for desired views (e.g., frontal, semi-profile), thereby creating a smaller, higher-quality subset of images for analysis.

The process 900 includes, upon extracting the second set of images, detecting a set of regions within the second set of images that represent one or more target anatomical structures of the subject body (910). For example, the feature engine 206 can fit a three-dimensional face mesh model onto an extracted frontal-view image and identify pre-defined groups of mesh vertices that correspond to regions such as the mental (chin) region, mandibular regions, and malar (cheek) regions.

In some implementations, the system can first generate a three-dimensional model of at least the portion of the subject body. The detection of the set of regions within the second set of images can then be performed using the generated three-dimensional model. For example, the processing engine 114 can generate a 3D face mesh from the extracted images, and the feature engine 206 can then use the known topology of the mesh to accurately detect target anatomical regions like the maxilla or mandible by referencing specific, pre-mapped vertex groups.

In some cases, detecting the set of regions within the second set of images can involve detecting a region within an oral cavity of the subject body, on a surface of the subject body, or both. For instance, a workflow configured via the experience studio can prompt the capture engine 108 to capture images of both the subject's face (a surface region) and their open mouth (an oral cavity region). The feature engine 206 can then detect facial landmarks from the surface images and anatomical structures like the uvula and soft palate from the oral cavity images.

The process 900 includes generating one or more anatomical features for at least one region of the set of regions that represent the target anatomical structures of the subject body (912). For example, using the identified mandibular and maxilla regions from the face mesh, the processing engine 114 can calculate a relative jaw depth difference, which serves as a generated anatomical feature.

The process 900 includes accessing one or more stored medical condition features associated with one or more medical conditions (914). For example, the prediction engine 210 can query a database to retrieve a threshold value for relative jaw depth difference that is clinically associated with retrognathia, a known indicator for obstructive sleep apnea.

The process 900 includes generating a predicted medical condition of the subject body, e.g., from among the one or more medical conditions, using the anatomical features and the stored medical condition features (916). For example, the prediction engine 210 can compare the subject's calculated jaw depth difference to the stored threshold for retrognathia. If the subject's value exceeds the threshold, the prediction engine 210 generates a prediction indicating an increased risk for obstructive sleep apnea.

In some cases, at least one of detecting the image features, detecting the regions, generating anatomical features, or generating the predicted medical condition can involve processing one or more items of input data using one or more machine learning models. For instance, the prediction engine 210 can utilize a dual-branch deep learning model, as shown in FIG. 7, to generate the predicted medical condition. In this example, one or more machine learning models are configured to process multi-channel input tensors comprising RGB image data and a corresponding anatomical mask to identify complex patterns indicative of a medical condition. A four-channel input tensor can be processed, which comprises a three-channel image of a portion of the subject body and a one-channel anatomical model corresponding to that portion.

In some implementations, generating the predicted medical condition can involve extracting a first value from the anatomical features and a second value from the stored medical condition features, and then generating the prediction based on a comparison. For example, the processing engine 114 can extract the calculated jaw depth measurement (the first value) and the prediction engine 210 can access the stored clinical threshold for retrognathia (the second value). The prediction engine 210 then generates the prediction based on whether the measured value exceeds the threshold. This comparison can involve a distance computation or one or more threshold checks.

In some cases, capturing the first set of images can include generating an estimated pose of the subject body and providing a prompt for adjustment based on the pose. For example, the capture engine 108 can run a live head pose estimator. If the subject's head is tilted beyond a roll tolerance, a visual prompt, such as ‘Please straighten your head,’ can be displayed on the user device 104 to guide the subject into an optimal position for data capture.

In some cases, the system can capture a set of responses to a questionnaire. The generation of the predicted medical condition can further use these responses. For example, the capture engine 108 can first present the screening questionnaire 504. The prediction engine 210 can then use the answers, in combination with the anatomical features, within a decision fusion module to generate a more comprehensive risk score.

The process 900 includes transmitting a signal indicating the generated predicted medical condition to a connected device (918). For example, the output engine 116 can transmit the generated prediction output, such as a risk score for obstructive sleep apnea, to the user device 104, where it is displayed to the user on the result screen 514.

In some cases, transmitting the signal indicating the generated predicted medical condition comprises presenting the information on a display device. For example, the output engine 116 can generate a graphical user interface, such as the result screen 514 or the result screen 556, that visually displays the prediction to the subject body 102 or to a clinician via the user device 104. This presentation can include a numerical risk score, a categorical classification (e.g., “low risk,” “high risk”), a graphical representation like a color-coded meter, or detailed reports as shown in the example CDS report of FIG. 10. The user interface can also provide explanatory text, visualizations of the key anatomical features that influenced the prediction, and actionable recommendations or next steps, such as an option to schedule a consultation with a healthcare provider.

In some implementations, the system can generate a treatment recommendation for the subject body using the predicted medical condition. The signal transmitted to the connected device can indicate this recommendation. For instance, if the prediction engine 210 predicts a high likelihood of OSA due to retrognathia, it may generate a recommendation for a consultation with a dentist for a potential oral appliance. The output engine 116 then transmits a signal that causes this recommendation to be displayed on the result screen 514 of the user device 104.

The processes 800 and 900 are illustrated as logical flow diagrams, each operation of which may be implemented in hardware, software, firmware, or a combination thereof. In various aspects of the described techniques, the order of operations may be different from that shown, and in some cases, one or more operations may be omitted or repeated. Furthermore, additional operations not explicitly shown may be included in the processes. For example, a process might omit a feature detection operation (e.g., operation 904). If a high-quality initial image is captured, a process could omit the operations of accessing feature thresholds and extracting a subset of images (e.g., operations 806 and 808), proceeding directly to detecting regions within that initial image. Similarly, a process could omit capturing questionnaire responses (as referenced in the description of process 900) and generate a prediction based solely on anatomical features derived from captured images. These variations are contemplated and fall within the scope of the techniques described herein.

In some implementations, a system such as the system 100 or the system 200, can be configured to detect craniofacial skeletal dysmorphologies, which can be anatomical contributors to medical conditions, such as OSA. Craniofacial skeletal dysmorphologies can cause various risks due to their role in narrowing a subject's airway. Conditions such as mandibular retrognathia, micrognathia, maxillary hypoplasia, and mandibular prognathism can be identified. To perform this detection, a model, which may be operated by the prediction engine 210, can be configured with a dual-branch architecture, e.g., processing different data. For example, the dual-branch architecture can split inputs into multi-view inputs, e.g., including images from the frontal view depicting the entire face, an image from the semi-profile view of the face depicting the facial regions below the eyes (e.g., the lower portion of the subject's chin), and images depicting the tongue as a proxy for the oral cavity. The prediction engine 210 can receive both frontal and semi-profile facial images captured by the capture engine 108. For each image view, a suitable engine, such as the processing engine 114 can compute a model, such as a dense 3D mesh, and construct a multi-channel input tensor. This tensor can include various data, such as the original RGB image, a landmark map derived from 2D fiducial points, and a normalized depth map, allowing the model to leverage texture, geometry, and depth information simultaneously. The features extracted from both views can be fused and passed to a classification head designed to identify specific skeletal dysmorphologies. The training of this, or other models, can incorporate fairness constraints to help reduce performance disparities across different demographic groups.

A system can be configured to detect functional orofacial abnormalities, which can be associated with impaired upper-airway function and heightened OSA risk. These abnormalities can include lip incompetence, open mouth posture, altered tongue posture, and other facial disproportions that may signal compromised neuromuscular coordination. Similar to the detection of skeletal dysmorphologies, a learning model, such as a deep learning model, can be configured to process multi-channel facial inputs, which can be generated by operations similar to those performed by the processing engine 114. In this case, the model can focus on cropped frontal orofacial regions. The model's classification head can be designed for multi-label classification to identify the presence of one or more functional abnormalities. The performance of such models can be evaluated using metrics such as the Area Under the Receiver Operating Characteristic curve (AUROC), sensitivity, specificity, and F1-score, which can be assessed for overall performance and stratified by demographic subgroups to monitor for fairness.

A system can be configured for the longitudinal assessment of skeletal and functional orofacial changes following OSA interventions, such as myofunctional therapy or orthodontic treatment. To quantify these changes, a model, such as a multi-view Siamese network, can be developed. This model can be configured to process paired pre-and post-treatment images, captured by a device such as the user device 104, in a multi-channel format that includes RGB, landmark, and depth information. A shared model backbone can encode features from both pre- and post-treatment images, and these features can be compared to emphasize directional change (e.g., improvement or regression). View-specific features from frontal and profile images can be fused into a unified representation, which is then passed to a classification head. This classification head can be configured to output a directional label for each condition, along with an associated probability score, providing an automated and interpretable method for tracking treatment response by linking image-based phenotypic changes to reductions in airway risk.

A system can be configured for the longitudinal assessment of a subject body's medical condition, which can be particularly useful for monitoring the progression of a condition or evaluating the effectiveness of a treatment intervention. Such a system can collect data over time, including images, videos, and data streams from one or more wearable devices, such as a smartwatch or continuous glucose monitor. For example, after an initial screening using the technologies described herein, such as those shown in FIGS. 5A-B and FIG. 6, a subject body 102 could be provided with a treatment, such as a CPAP device or myofunctional therapy. The capture engine 108 can be configured to periodically capture new images of the subject's craniofacial region or oral cavity, or to receive health data from wearable devices. The processing engine 114 can then analyze this longitudinal data to detect changes in the anatomical features or health metrics. A model, such as a multi-view Siamese network as previously discussed or other network, can be used to compare pre- and post-treatment images to quantify directional changes, such as improvements in jaw alignment or reductions in facial swelling. Based on these tracked changes, the prediction engine 210 can generate updated risk scores or assessments, and the output engine 116 can provide recommendations for adjustments to the treatment plan, such as modifying CPAP pressure settings or suggesting follow-up consultations.

In some implementations, the system 100 or the system 200, can be configured to screen for Obstructive Sleep Apnea (OSA) in pediatric subjects. The analysis can be adapted to recognize specific anatomical markers and symptoms that manifest differently across various pediatric age groups. For example, the system can be configured with distinct feature sets and corresponding medical condition features for infants and young children (e.g., ages 0-5), school-age children (e.g., ages 5-12), and adolescents (e.g., ages 12 and above). A key indicator for pediatric OSA that the system can be configured to detect is “adenoid facies,” which can result from chronic mouth breathing due to enlarged adenoids. The detection of adenoid facies can involve a multi-faceted analysis of craniofacial features.

For instance, the processing engine 114 can be configured to analyze facial proportions to detect a long, narrow face, which can be characteristic of adenoid facies. This can involve calculating a ratio of the lower facial height (e.g., the distance from the base of the nose to the chin) to the total facial height and comparing it against age-specific normative data. Other manifestations of adenoid facies can be detected using various sub-operations. The system can be configured to detect an open mouth posture by analyzing the distance between the lips in a resting state. To identify sunken eyes or dark periorbital circles, the system can analyze pixel color and intensity in the periorbital region. Narrow or pinched nostrils can be identified from upward-facing images by using image segmentation to measure nostril area relative to the overall nasal structure. Furthermore, analysis of oral cavity images can be used to detect a high-arched palate, and analysis of habitual occlusion images can be used to identify crowded or misaligned teeth, which can be further indicators of pediatric OSA. The prediction engine 210 can then synthesize these findings to generate a risk score specifically tailored for pediatric OSA.

In some implementations, the system 100 or the system 200, can be configured to incorporate multi-modal data streams for generating a medical prediction. In addition to, or instead of, visual data from images and video, the system can be configured to capture and analyze audio or voice data. For example, the capture engine 108 can be configured to record a subject's voice, such as during speech or while producing sustained vowel sounds. The processing engine 114 can then analyze these voice samples to extract a set of acoustic features. These features can include, but are not limited to, pitch, jitter (frequency variations), shimmer (amplitude variations), and harmonic-to-noise ratio. The prediction engine 210 can then access stored medical condition features that correlate specific acoustic patterns with an increased risk of OSA. For instance, signs of vocal cord inflammation or altered vocal tract resonance, reflected in the acoustic features, can be indicative of airway obstruction or sleep-disordered breathing. This voice-based analysis can be used as an independent input for prediction or fused with features derived from visual and questionnaire data within the decision fusion module to generate a more comprehensive and robust assessment of a subject's OSA risk.

In some implementations, the system 100 or the system 200, can be configured with a specific multi-modal AI architecture to generate a predicted medical condition. Such an architecture, which may be referred to as an OSAFusionModel, can be designed to process distinct data types using specialized network backbones within a multi-branch structure. For example, a first branch of the model can be configured to process visual data, such as craniofacial imagery, using a convolutional neural network (CNN) based architecture. A CNN backbone can be well-suited for extracting spatial features and patterns from images. A second branch can be configured to process non-visual, categorical data, such as responses from a sleep health questionnaire. This second branch can utilize a Transformer-based architecture, which can be effective at capturing complex relationships and contextual dependencies between different categorical inputs, such as the answers to various health-related questions. The feature outputs from each branch, representing learned patterns from both the visual and categorical data, can then be fused, for example through concatenation or another suitable fusion technique. This fused representation can then be passed to one or more classification heads to generate a final prediction, thereby leveraging the complementary strengths of both CNNs for image analysis and Transformers for structured data analysis.

A system can be configured to construct and process a feature-rich multi-channel input tensor to enhance the analysis of visual data, such as craniofacial images. In some implementations, this can be a five-channel input tensor designed to provide a deep learning model with a unified representation that captures textural, geometric, and depth information simultaneously. Such a tensor can be constructed by concatenating multiple data layers. For instance, the first three channels can comprise a standard RGB color image of the subject. The fourth channel can be a Gaussian landmark map, generated by placing Gaussian distributions centered at the locations of key 2D fiducial landmarks on the face. This landmark map can provide the model with an explicit representation of the facial geometry. The fifth channel can be a normalized depth map, which can be derived from a model (e.g., a face mesh) fitted to the image, providing crucial information about the three-dimensional structure of the craniofacial complex. By combining these channels, the system can feed the model an input that concurrently encodes the subject's appearance, the precise location of key anatomical points, and the relative depth of different facial regions.

In some implementations, the training process for a machine learning model of the system 100 or the system 200, can incorporate fairness constraints to actively mitigate AI bias and reduce performance disparities across different demographic groups. This can be achieved by modifying the model's loss function during the training phase. For example, in addition to a primary loss component that measures the model's prediction accuracy (such as cross-entropy loss), one or more fairness-related penalty terms can be added. These penalty terms can be configured to measure and penalize discrepancies in key performance metrics, such as the true positive rate or false positive rate, between different subgroups defined by attributes like age, sex, or ethnicity. By including these fairness constraints in the overall loss calculation, the optimization process can be guided not only to maximize accuracy but also to minimize bias. This can encourage the model to learn representations that are robust and equitable, promoting demographic parity or equalized odds in its predictions.

In this specification the term ‘engine’ is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The subject matter and the actions and operations described in this specification can be implemented in digital electronic circuitry, in tangibly-embodied computer software or firmware, in computer hardware, including the structures disclosed in this specification and their structural equivalents, or in combinations of one or more of them. The subject matter and the actions and operations described in this specification can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively or in addition, the carrier can be an artificially-generated propagated signal, e.g., a machine-generated electrical, optical, or electromagnetic signal, that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term ‘data processing apparatus’ encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random access memory or both. The essential elements of a computer are a central processing unit for executing instructions and one or more memory devices for storing instructions and data. The central processing unit and the memory can be supplemented by, or incorporated in, special-purpose logic circuitry.

Generally, a computer will also include, or be operatively coupled to, one or more mass storage devices, and be configured to receive data from or transfer data to the mass storage devices. The mass storage devices can be, for example, magnetic, magneto-optical, or optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech, or tactile feedback or responses; and input from the user can be received in any form, including acoustic, speech, tactile, or eye tracking input, including touch motion or gestures, or kinetic motion or gestures or orientation motion or gestures. In addition, a computer can interact with a user by sending documents to and receiving documents from a device that is used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

This specification uses the term ‘configured to’ in connection with systems, apparatus, and computer program components. That a system of one or more computers is configured to perform particular operations or actions means that the system has installed on it software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

The subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network.

The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
capturing a first set of images depicting at least a portion of an oral cavity region of a subject body;
detecting a set of image features associated with the oral cavity region within the first set of images;
accessing one or more stored feature thresholds;
extracting, from the first set of images and using the stored feature thresholds, a second set of images that satisfy one or more of the stored feature thresholds;
upon extracting the second set of images, detecting a set of regions within the second set of images that represent one or more target anatomical structures within the oral cavity region of the subject body;
generating one or more anatomical features for at least one region of the set of regions that represent the target anatomical structures of the subject body;
accessing one or more stored obstructive sleep apnea features that indicate obstructive sleep apnea;
generating a prediction indicating a likelihood that the subject body suffers from obstructive sleep apnea using (i) the anatomical features for the at least one region of the oral cavity of the subject body and (ii) the stored obstructive sleep apnea features; and
transmitting a signal indicating the generated prediction of obstructive sleep apnea to a connected device.

2. The method of claim 1, wherein capturing the first set of images depicting at least the portion of the oral cavity region of the subject body comprises:
capturing the first set of images using a handheld image capture device.

3. The method of claim 2, wherein capturing the first set of images using the handheld image capture device comprises:
capturing the first set of images using a smartphone.

4. The method of claim 1, wherein capturing the first set of images depicting at least the portion of the oral cavity region of the subject body comprises:
capturing an image of a surface region of a head of the subject body, wherein the method comprises:
detecting a second set of image features associated with the head of the subject body, wherein accessing the one or more stored feature thresholds comprises:
accessing a stored feature threshold corresponding to a head feature, wherein extracting the second set of images that satisfy one or more of the stored feature thresholds comprises:
extracting (i) a first image that indicates a first feature that satisfies a first stored feature threshold associated with the oral cavity region of the subject body and (ii) a second image that indicates a second feature that satisfies a second stored feature threshold associated with the head of the subject body.

5. The method of claim 1, comprising:
generating a three-dimensional model of at least the surface region of the head of the subject body, wherein detecting the set of regions within the second set of images comprises detecting the set of regions using the generated three-dimensional model.

6. The method of claim 5, wherein detecting the set of regions within the second set of images comprises:
detecting a lower jaw region and an upper jaw region using the generated three-dimensional model.

7. The method of claim 1, wherein detecting the set of image features associated with the oral cavity region within the first set of images comprises:
detecting a subregion of the oral cavity region of the subject body, wherein the subregion includes at least one of an upper teeth region, a lower teeth region, a back of the mouth region, or a tongue region.

8. The method of claim 7, wherein accessing the one or more stored feature thresholds comprises:
accessing an axis of symmetry feature threshold indicating a degree of symmetry within one or more subregions of the oral cavity region of the subject body, wherein extracting the second set of images that satisfy one or more of the stored feature thresholds comprises:
extracting an image that includes a subregion within a threshold percentage of vertical alignment.

9. The method of claim 1, wherein detecting the set of image features associated with the oral cavity region within the first set of images comprises using an image segmentation library to identify a plurality of segmented regions within the oral cavity region, and wherein extracting the second set of images that satisfy one or more of the stored feature thresholds comprises:
extracting an image in which the image segmentation library identifies at least three well-defined regions selected from a group consisting of an upper teeth region, a lower teeth region, a back of the mouth region, and a tongue region, wherein a vertical axis of symmetry for each of the at least three well-defined regions is substantially in alignment.

10. The method of claim 1, further comprising screening for one or more medical conditions comorbid with obstructive sleep apnea, wherein screening for the one or more medical conditions comprises:
capturing a second set of images depicting a motor task performed by at least one hand of the subject body;
detecting, from the second set of images, a plurality of hand features corresponding to movements of the at least one hand;
generating one or more movement metrics based on the plurality of hand features; and
generating a prediction indicating a likelihood of a neurodegenerative condition based on the one or more movement metrics.

11. The method of claim 10, wherein the motor task comprises repeatedly touching a tip of an index finger to a tip of a thumb, and wherein generating the one or more movement metrics comprises calculating a frequency of contact between the tip of the index finger and the tip of the thumb.

12. The method of claim 1, further comprising screening for a cardiovascular condition comorbid with obstructive sleep apnea, wherein screening for the cardiovascular condition comprises:
detecting, from the first set of images, a region corresponding to an earlobe of the subject body;
analyzing the region corresponding to the earlobe to detect a presence of a diagonal crease; and
generating a prediction indicating an increased risk of coronary artery disease in response to detecting the presence of the diagonal crease.

13. The method of claim 1, further comprising screening for a potential past stroke comorbid with obstructive sleep apnea, wherein screening for the potential past stroke comprises:
identifying a plurality of fiducial landmarks on a face of the subject body within the first set of images;
calculating a degree of facial symmetry using the plurality of fiducial landmarks; and
generating a prediction indicating a likelihood of a past stroke in response to determining that the degree of facial symmetry is below a symmetry threshold.

14. One or more non-transitory computer storage media encoded with computer program instructions that when executed by one or more computers cause the one or more computers to perform operations comprising:
capturing a first set of images depicting at least a portion of an oral cavity region of a subject body;
detecting a set of image features associated with the oral cavity region within the first set of images;
accessing one or more stored feature thresholds;
extracting, from the first set of images and using the stored feature thresholds, a second set of images that satisfy one or more of the stored feature thresholds;
upon extracting the second set of images, detecting a set of regions within the second set of images that represent one or more target anatomical structures within the oral cavity region of the subject body;
generating one or more anatomical features for at least one region of the set of regions that represent the target anatomical structures of the subject body;
accessing one or more stored obstructive sleep apnea features that indicate obstructive sleep apnea;
generating a prediction indicating a likelihood that the subject body suffers from obstructive sleep apnea using (i) the anatomical features for the at least one region of the oral cavity of the subject body and (ii) the stored obstructive sleep apnea features; and
transmitting a signal indicating the generated prediction of obstructive sleep apnea to a connected device.

15. A system comprising:
one or more computers and one or more storage devices on which are stored instructions that are operable, when executed by the one or more computers, to cause the one or more computers to perform operations:
capturing a first set of images depicting at least a portion of an oral cavity region of a subject body;
detecting a set of image features associated with the oral cavity region within the first set of images;
accessing one or more stored feature thresholds;
extracting, from the first set of images and using the stored feature thresholds, a second set of images that satisfy one or more of the stored feature thresholds;
upon extracting the second set of images, detecting a set of regions within the second set of images that represent one or more target anatomical structures within the oral cavity region of the subject body;
generating one or more anatomical features for at least one region of the set of regions that represent the target anatomical structures of the subject body;
accessing one or more stored obstructive sleep apnea features that indicate obstructive sleep apnea;
generating a prediction indicating a likelihood that the subject body suffers from obstructive sleep apnea using (i) the anatomical features for the at least one region of the oral cavity of the subject body and (ii) the stored obstructive sleep apnea features; and
transmitting a signal indicating the generated prediction of obstructive sleep apnea to a connected device.

16. A method comprising:
capturing a first set of images depicting at least a portion of a subject body;
detecting a set of image features within the first set of images;
accessing one or more stored feature thresholds;
extracting, from the first set of images and using the stored feature thresholds, a second set of images that satisfy one or more criteria corresponding to the stored feature thresholds;
upon extracting the second set of images, detecting a set of regions within the second set of images that represent one or more target anatomical structures of the subject body;
generating one or more anatomical features for at least one region of the set of regions that represent the target anatomical structures of the subject body;
accessing one or more stored medical condition features associated with one or more medical conditions;
generating a predicted medical condition of the subject body, from among the one or more medical conditions, using the anatomical features and the stored medical condition features; and
transmitting a signal indicating the generated predicted medical condition to a connected device.

17. The method of claim 16, comprising:
generating a three-dimensional model of at least the portion of the subject body, wherein detecting the set of regions within the second set of images comprises detecting the set of regions using the generated three-dimensional model.

18. The method of claim 16, wherein detecting the set of regions within the second set of images that represent the one or more target anatomical structures of the subject body comprises:
detecting a region within an oral cavity of the subject body, on a surface of the subject body, or both within the oral cavity of the subject body and on the surface of the subject body.

19. The method of claim 16, wherein at least one of (i) detecting the set of image features, (ii) detecting the set of regions, (iii) generating one or more anatomical features, or (iv) generating the predicted medical condition, comprises:
processing one or more items of input data using one or more machine learning models.

20. The method of claim 19, wherein processing the one or more items of input data using the one or more machine learning models comprises:
processing a four-channel input tensor to generate the predicted medical condition, wherein the four-channel input tensor comprises a three-channel image of a portion of the subject body and a one-channel anatomical model corresponding to the portion of the subject body.

21. The method of claim 16, wherein generating the predicted medical condition comprises:
extracting at least a first value from the anatomical features and at least a second value from the stored medical condition features; and
generating the predicted medical condition based on a comparison of the first value and the second value.

22. The method of claim 21, wherein the comparison of the first value and the second value comprises at least one of a distance computation or one or more threshold checks.

23. The method of claim 16, comprising:
generating a treatment recommendation for treating the subject body using the predicted medical condition, wherein transmitting the signal indicating the generated predicted medical condition to the connected device comprises:
transmitting a signal that indicates the generated treatment recommendation for treating the subject body.

24. The method of claim 16, wherein capturing the first set of images comprises:
generating an estimated pose of the subject body within the first set of images; and
providing a prompt to indicate an adjustment for the subject body based on the estimated pose.

25. The method of claim 16, further comprising:
capturing a set of responses to a questionnaire; and
generating the predicted medical condition further uses the set of responses to the questionnaire.

26. The method of claim 16, wherein the set of regions consists of a single region.

27. The method of claim 16, wherein the second set of images consists of a single image.

28. The method of claim 16, wherein the set of regions within the second set of images represent at least one of a mandibular region, a malar region, a sub-malar region, a mental region, or a periorbital region.

29. The method of claim 16, wherein the set of regions within the second set of images represent a jaw of the subject body.

30. The method of claim 16, wherein the set of regions within the second set of images represent one or more regions of an oral cavity of the subject body.
